(19) United States
(12) Patent Application Publication
(10) Pub. No.: US 2020/0397728 A1
(43) Pub. Date: Dec. 24, 2020

(54) PHARMACEUTICAL FORMULATION FOR CARGLUMIC ACID

(71) Applicant: RECORDATI S.p.A., Milano (IT)

(72) Inventors: Alberto MATTEI, Milano (IT); Marco BARCHIELLI, Milano (IT); Stefano SELVA, Milano (IT); Eduardo BOLZAN, Milano (IT)

(21) Appl. No.: 16/887,701

(22) Filed: May 29, 2020

Related U.S. Application Data
(60) Provisional application No. 62/854,661, filed on May 30, 2019.

(51) Int. Cl. A61K 31/17; A61K 31/133; A61K 9/46; A61K 47/26; A61K 47/14; A61K 47/38; A61K 47/02; A61K 9/20

(52) U.S. Cl. CPC A61K 31/17; A61K 31/133; A61K 9/0007; A61K 9/20; A61K 47/14; A61K 47/38; A61K 47/02; A61K 47/26

(57) ABSTRACT

The present disclosure relates to a pharmaceutical formulation comprising carglumic acid, tromethamine, and one or more pharmaceutically acceptable excipients. The formulation of the present disclosure can be useful for treating hyperammonaemia

### CROSS-REFERENCE TO RELATED APPLICATIONS

This application claims priority under 35 U.S.C. 119(c) to U.S. Provisional Application Ser. No. 62/854,661, filed May 30, 2019, which is herein incorporated by reference in its entirety.

### FIELD OF THE DISCLOSURE

The present invention relates to a pharmaceutical formulation comprising carglumic acid, tromethamine, and one or more pharmaceutically acceptable excipients. In particular, the disclosure relates to a carglumic acid formulation for the treatment of hyperammonaemia.

### BACKGROUND OF THE DISCLOSURE

Carglumic acid, whose chemical formula is reported below, is an active pharmaceutical agent that is used for the treatment of hyperammonaemia (high blood levels of ammonia).

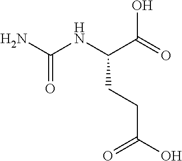 [SMILES: NC(=O)N[C@@H](CCC(=O)O)C(=O)O]

Carglumic acid is marketed in the EU under the trademark Carbaglu® in the form of tablets that must be dispersed in water and ingested immediately or administered by fast push through a syringe via a nasogastric tube, generally in case of hospitalized patients or patients who are not able to swallow.

Carglumic acid is highly hygroscopic and suffers some instability problems. For instance, unopened Carbaglu® containers should be tightly closed and stored at 2 to 8° C. After its first opening, the container must be stored at a temperature above the refrigerated temperature but below 30° C.; furthermore, any unused tablets must be discarded after one month from the first opening.

Processes for manufacturing tablets containing carglumic acid by direct compression are disclosed in EP2777696 and CN 105056246.

Lyophilized formulation of carglumic acid is disclosed in WO 2018/095848, which is hereby incorporated by reference in their entireties.

Due to long term instability of carglumic acid, there is a need for a stable formulation that can be easily administered such that patient compliances can be improved.

### SUMMARY OF THE DISCLOSURE

The present disclosure relates to a pharmaceutical formulation comprising carglumic acid. In one embodiment, the pharmaceutical formulation comprises (a) carglumic acid or a pharmaceutically acceptable salt thereof (b) tromethamine; and (c) one or more pharmaceutically acceptable excipients.

In one embodiment of the pharmaceutical formulation as disclosed herein, carglumic acid or a pharmaceutically acceptable salt thereof and tromethamine are present at a molar ratio of from about 1:1 to about 1.5:1. In one embodiment, carglumic acid or a pharmaceutically acceptable salt thereof and tromethamine are present at a molar ratio from about 1:1 to about 1.2:1. In one embodiment, carglumic acid or a pharmaceutically acceptable salt thereof and tromethamine are present at a molar ratio of about 1.16:1.

In one embodiment of the pharmaceutical formulation as disclosed herein, comprises one or more pharmaceutically acceptable excipients.

In one embodiment of the pharmaceutical formulation as disclosed herein, the one or more pharmaceutically acceptable excipients is a filler. In one embodiment, the filler is selected from sugars, polyalcohols, amino acids, polymers, polysaccharides, inorganic salts, silica, or combinations thereof. In one embodiment, the filler is selected from glucose, mannose, maltose, sucrose, lactose, sorbitol, mannitol, maltitol, xylitol, glycine, polyvinylpyrrolidone (Crospovidone), Poly(l-vinylpyrrolidone-co-vinyl acetate) (Copovidone), dextran, sodium phosphate, potassium phosphate, sodium chloride, silicon dioxide, or combinations thereof. In some embodiments, the filler is sorbitol, mannitol, maltitol, crospovidone, copovidone, silicon dioxide, or combinations thereof. In one embodiment, the filler is Pharmaburst®.

In one embodiment of the pharmaceutical formulation as disclosed herein, the one or more pharmaceutically acceptable excipients is an effervescent agent. In one embodiment, the effervescent agent is selected from alkali metal bicarbonate, an alkaline earth metal bicarbonate, an alkali metal carbonate, an organic carbonate, or combinations thereof. In some embodiments, the effervescent agent is selected from ammonium bicarbonate, calcium bicarbonate, lithium bicarbonate, magnesium bicarbonate, potassium bicarbonate, sodium bicarbonate, arginine carbonate, ammonium carbonate, calcium carbonate, lysine carbonate, potassium magnesium carbonate, sodium carbonate, sodium glycine carbonate, sodium sesquicarbonate, zinc carbonate, or combinations thereof. In one embodiment, the effervescent agent is sodium carbonate, sodium bicarbonate, or combinations thereof. In one embodiment, the effervescent agent is sodium bicarbonate.

In one embodiment of the pharmaceutical formulation as disclosed herein, the one or more pharmaceutically acceptable excipients is a lubricant. In one embodiment, the lubricant is stearic acid, palmitic acid, calcium hydroxide, talc, corn starch, sodium stearyl fumarate, sodium stearate, magnesium stearate, zinc stearate, aluminum stearate, leucine, polyethylene glycol, glyceryl behenate, colloidal silicon dioxide, hydrogenated vegetable oil, mineral oil, or waxes. In one embodiment, the lubricant is magnesium stearate or sodium stearyl fumarate. In some embodiments, the lubricant is sodium stearyl fumarate.

In one embodiment of the pharmaceutical formulation as disclosed herein, the one or more pharmaceutically acceptable excipients is a sweetener. In one embodiment, the sweetener is selected from sugars or sugar alcohols. In one embodiment, the sweetener is selected from aspartame, ammonium glycyrrhizinate, sucralose, shaccarin sodium, sucrose, glucose, lactose, fructose, sorbitol, xylitol, or erythritol. In some embodiments, the sweetener is sucralose.

In one embodiment of the pharmaceutical formulation as disclosed herein, the one or more pharmaceutically acceptable excipients is a binder. In one embodiment, the binder is selected from celluloses, cellulose ethers, cellulose esters, tricalcium phosphate, povidone, copovidone, pregelatinized starch, dextrin, gelatin, maltodextrin, starch, zein, acacia, alginic acid, carbomers, cross-linked polyacrylates, polymethacrylates, guar gum, hydrogenated vegetable oil, magnesium aluminum silicate, or sodium alginate. In some embodiments, the binder is selected from methylcellulose, carboxymethylcellulose, hydroxypropyl cellulose, ethylcellulose, hydroxypropyl methylcellulose, or hydroxyethyl cellulose. In one embodiment, the binder is hydroxypropyl methylcellulose (HPMC).

In one embodiment of the pharmaceutical formulation as disclosed herein, the one or more pharmaceutically acceptable excipients is a wetting agent. In one embodiment, the wetting agent is selected from sucrose palmitate, polyethylene glycol-polypropylene glycol copolymer, metal alkyl sulfate, sodium lauryl sulfate, polyoxyethylene sorbitan fatty acid ester, polyoxyethylene alkyl ether, polyethylene glycol, polyoxyethylene castor oil derivatives, docusate sodium, quaternary ammonium amine compounds, sugar esters of fatty acids, polyethoxylated fatty acid esters, glycerides of fatty acids, and polyglycolized glycerides. In some embodiments, the wetting agent is polyethylene glycol or sucrose palmitate. In one embodiment, the wetting agent is sucrose palmitate.

In one embodiment of the pharmaceutical formulation as disclosed herein, the one or more pharmaceutically acceptable excipients is a glidant. In one embodiment, the glidant is selected from powdered cellulose, colloidal silicon dioxide, calcium silicate, magnesium trisilicate, talc, corn starch, or a combination thereof. In some embodiments, the glidant is colloidal silicon dioxide.

In one embodiment of the pharmaceutical formulation as disclosed herein, the formulation is in the form of a tablet. In one embodiment, the tablet has a disintegration time of less than 3 minutes. In other embodiments, the tablet has a disintegration time of less than or equal to 90 seconds.

In one embodiment of the tablet pharmaceutical formulation as disclosed herein, the tablet has a dissolution profile characterized by at least 85% dissolution in pH 1.2 hydrochloric acid medium at 37° C.±0.5° C. in no more than 15 minutes as measured by high-performance liquid chromatography at 200 nm.

In one embodiment of the tablet pharmaceutical formulation as disclosed herein, the tablet has a dissolution profile characterized by at least 85% dissolution in pH 4.5 sodium acetate buffer at 37° C.±0.5° C. in no more than 15 minutes as measured by high-performance liquid chromatography at 200 nm.

In one embodiment of the tablet pharmaceutical formulation as disclosed herein, the tablet has a dissolution profile characterized by at least 85% dissolution in pH 6.8 potassium phosphate buffer at 37° C.±0.5° C. in no more than 15 minutes as measured by high-performance liquid chromatography at 200 nm.

In one embodiment of the pharmaceutical formulation as disclosed herein, the formulation comprises less than 0.5% of Impurity 1. In one embodiment, the formulation comprises less than 0.2% of Impurity 1. In other embodiments, the formulation comprises less than or equal to 0.10% of Impurity 1.

In one embodiment of the pharmaceutical formulation as disclosed herein, the formulation comprises less than or equal to 0.10% of Impurity 1 after the formulation is stored at 25° C. at 60% relative humidity (RH) for 3 months.

In one embodiment of the pharmaceutical formulation as disclosed herein, the formulation comprises less than or equal to 0.10% of Impurity 1 after the formulation is stored at 30° C. at 65% RH for 3 months.

In one embodiment of the present disclosure, a unit dose comprising (a) about 100 mg to about 1000 mg of carglumic acid or a pharmaceutically acceptable salt thereof; (b) tromethamine; and (c) one or more pharmaceutically acceptable excipients is disclosed.

In one embodiment of the unit dose formulation, the formulation comprises about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, or about 850 mg of carglumic acid or a pharmaceutically acceptable salt thereof.

In some embodiments, the unit dose formulation comprises about 200 mg of carglumic acid or a pharmaceutically acceptable salt thereof. In one embodiment of the unit dose formulation comprising about 200 mg of carglumic acid, the formulation comprises about 90 mg to about 150 mg of tromethamine. In some embodiments, the unit dose comprising about 200 mg of carglumic acid, comprises about 110 mg of tromethamine.

In one embodiment of the unit dose formulation comprising about 200 mg of carglumic acid, the formulation comprises about 100 mg to about 200 mg of Pharmaburst®. In one embodiment, the unit dose comprising about 200 mg of carglumic acid, comprises about 140 mg of Pharmaburst®.

In some embodiments, the unit dose formulation comprises about 800 mg of carglumic acid or a pharmaceutically acceptable salt thereof. In one embodiment of the unit dose formulation comprising about 800 mg of carglumic acid, the unit dose comprises about 400 mg to about 500 mg of tromethamine. In one embodiment of the unit dose formulation comprising about 800 mg of carglumic acid, the unit dose comprises about 440 mg of tromethamine.

In one embodiment of the unit dose formulation comprising about 800 mg of carglumic acid, the unit dose comprises about 500 mg to about 600 mg of Pharmaburst®. In one embodiment of the unit dose formulation comprising about 800 mg of carglumic acid, the unit dose comprises about 561 mg of Pharmaburst®.

In one embodiment of the present disclosure, a method of treating hyperammonaemia is provided. In one embodiment, a method of treating hyperammonaemia comprises administering an effective amount of any one of pharmaceutical formulations as disclosed herein, to a patient in need thereof. In one embodiment, a method of treating hyperammonaemia comprises administering an effective amount of any one of unit doses as disclosed herein, to a patient in need thereof.

### BRIEF DESCRIPTION OF THE DRAWINGS

FIG. 1 shows dissolution profile of two different batches Carbaglu® Market tablets and the carglumic acid tablet of Table 22.

FIG. 2 shows comparison of solubility between Carbaglu® Market tablets and the carglumic acid tablet of Table 22, at a high dose simulation.

FIG. 3 shows comparison of solubility between Carbaglu® Market tablets and the carglumic acid tablet of Table 22, at a low dose simulation.

FIG. 4 shows solubility gap between Carbaglu® Market tablets and the carglumic acid tablet of Table 22.

FIG. 5 shows comparison of solubility between Carbaglu® Market tablets and the carglumic acid tablet of Table 22, in an acidic media.

FIG. 6 shows HPLC chromatogram of a D-carglumic acid reference solution spiked at the specification limit of 0.1%. D-carglumic acid.

FIG. 7 shows HPLC chromatogram of a carglumic acid tablet having the composition of Table 22 which was spiked with 0.10% D-carglumic acid.

FIG. 8 shows HPLC chromatogram of 200 mg carglumic acid tablet having the composition in Table 22 with hardness value of 50 N.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The terms “tromethamine,” “trometamol,” and “TRIS,” which can be used interchangeably, all refers to tris(hydroxymethyl)aminomethane.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

As used herein, a “pediatric population” refers to human patients who are 18 years old or younger.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

“ICH conditions” as used herein refers to the thermohygrometric conditions of storage of Drug Products that are intended for already marketed products or submissions of new Marketing Authorizations (MA), outlined by the International Council on Harmonisation (ICH) guidelines.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof

The following description includes information that may be useful in understanding the present invention. It is not an admission that any of the information provided herein is prior art or relevant to the presently claimed inventions, or that any publication specifically or implicitly referenced is prior art.

Formulations of the Present Disclosure

The Carbaglu® tablet currently available on the market (“Carbaglu® Market”) has the composition as shown in Table A.

TABLE A
Carbaglu ® Market Formulation

| | |
|---|---|
| Carglumic Acid | 200.00 mg |
| Mycrocristalline Cellulose | 270.00 mg |
| Sodium Lauryl Sulfate | 0.50 mg |
| Hypromellose | 4.00 mg |
| Croscarmellose Sodium | 19.00 mg |
| Sodium Stearyl Fumarate | 5.00 mg |
| Colloidal Silica Dioxide | 1.50 mg |

The Carbaglu® Market formulation presents the following limitations for the patient:

a) Presence of insoluble/swellable excipients used in the formulation (i.e. binder such as microcrystalline cellulose, MCC) which once dispersed in water, do not solubilize so to create a fluffy, high volume sediment on the bottom of the glass. Therefore it requires the patient to rinse at least one time more the glass in order to ensure a complete dose administration and could create a “sandy” perception to the patients during swallowing.

b) Sour and bitter taste, directly linked to the active substance as a consequence of its solubilization.

c) A partial dispersion of not solubilized carglumic acid when high dosages are administered through a limited amount of water (limitation due to the intrinsic solubility of the carglumic acid at its natural pH)

d) Risk of clogging of devices such as nasogastric catheters, approved for emergency treatments, due to the presence of high insoluble residues.

Thus, there was a need to improve the current Carbaglu® Market formulation to address the above noted limitations.

Pharmaceutically Acceptable Excipients

In certain embodiments, the pharmaceutical formulations of the present disclosure may additionally contain other adjunct components conventionally found in pharmaceutical formulations, at their art-established usage levels. For example, the pharmaceutical formulation of the present disclosure may contain additional, compatible, materials such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the pharmaceutical formulations presented herein can be formulated for administration by a variety of means including orally, parenterally, by inhalation spray, topically, or rectally. The term parenteral as used here includes subcutaneous, intravenous, intramuscular, and intraarterial injections with a variety of infusion techniques. Intraarterial and intravenous injection as used herein includes administration through catheters.

The formulations disclosed herein can be formulated in accordance with the routine procedures adapted for desired administration route. Accordingly, the formulations disclosed herein can take such forms as suspensions, solutions or emulsions in oily or aqueous vehicles, and can contain formulatory agents such as suspending, stabilizing and/or dispersing agents. The formulations can be prepared for implantation or injection. Thus, for example, carglumic acid can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, carglumic acid formulation of the present disclosure is in a tablet form. In one embodiment, the carglumic acid tablet formulation is to be administered by first dissolving the tablet in water.

In one embodiment, the formulation of the present disclosure comprises a buffering agent. The term “buffering agent” as used herein refers to a weak acid or base used to adjust the pH value of a water solution of a formulation to be at a specific pH. A buffering agent can increase the solubility of the active substance. In one embodiment, the buffering agent is selected from citrates, phosphates, borates, acetates, sodium hydroxide, glycine, triethanoloamine, tromethamine, meglumine, or salts thereof or combinations thereof. In one embodiment, the buffering agent is selected from sodium acetate, sodium citrate, sodium borate, one or more sodium salts of phosphoric acid, one or more potassium salts of phosphoric acid, sodium bicarbonate, tromethamine, meglumine or combinations thereof. In one embodiment, the buffering agent is selected from an alkali metal salt, alkali-earth metal salt, or an ammonium salt of: citric acid, ascorbic acid, maleic acid, sorbic acid, succinic acid, benzoic acid, phosphoric acid, carbonic acid, sulfuric acid, nitric acid, boric acid, or silicic acid. In one embodiment, the buffering agent is tromethamine.

In one embodiment, the formulation of the present disclosure comprises tromethamine. In one embodiment, tromethamine acts as a buffering agent to increase the solubility of the active substance.

In one embodiment, the formulation of the present disclosure comprises tromethamine and carglumic acid or a pharmaceutically acceptable salt thereof in molar ratio of about 1:1 to about 1:1.5. That is, the molar ratio of tromethamine and carglumic acid or a pharmaceutically acceptable salt thereof can be about 1:1.0, about 1:1.05, about 1:1.1, about 1:1.15, about 1:1.2, about 1:1.25, about 1:1.3, about 1:1.35, about 1:1.4, about 1:1.45, or about 1:1.5. In one embodiment, the molar ratio of tromethamine and carglumic acid or a pharmaceutically acceptable salt thereof is about 1:1.13, about 1:1.14, about 1:1.15, about 1:1.16, about 1:1.17, or about 1:1.18. In one embodiment, the molar ratio of tromethamine and carglumic acid or a pharmaceutically acceptable salt thereof is about 1:1.16.

In one embodiment, the formulation of the present disclosure comprises a filler. In one embodiment, the filler is selected from sugars, polyalcohols, amino acids, polymers, polysaccharides, inorganic salts, silica, or combinations thereof. In one embodiment, the filler is selected from glucose, mannose, maltose, sucrose, lactose, sorbitol, mannitol, maltitol, xylitol, glycine, polyvinylpyrrolidone (Crospovidone), Poly(l-vinylpyrrolidone-co-vinyl acetate) (Copovidone), dextran, sodium phosphate, potassium phosphate, sodium chloride, silicon dioxide, or combinations thereof. In one embodiment, the filler is sorbitol, mannitol, maltitol, crospovidone, copovidone, silicon dioxide, or combinations thereof. In one embodiment, the filler is Pharmaburst® (SPI Pharma).

In one embodiment, the filler is water-soluble.

In one embodiment, the formulation of the present disclosure comprises an effervescent agent. In one embodiment, the effervescent agent effervescent agent is selected from alkali metal bicarbonate, an alkaline earth metal bicarbonate, an alkali metal carbonate, an organic carbonate, or combinations thereof. In one embodiment, the effervescent agent is selected from ammonium bicarbonate, calcium bicarbonate, lithium bicarbonate, magnesium bicarbonate, potassium bicarbonate, sodium bicarbonate, arginine carbonate, ammonium carbonate, calcium carbonate, lysine carbonate, potassium magnesium carbonate, sodium carbonate, sodium glycine carbonate, sodium sesquicarbonate, zinc carbonate, or combinations thereof. In one embodiment, the effervescent agent is sodium carbonate, sodium bicarbonate, or combinations thereof. In one embodiment, the effervescent agent is Effersoda® (SPI Pharma). In one embodiment, the effervescent agent is sodium bicarbonate.

In one embodiment, the formulation of the present disclosure comprises a lubricant. In one embodiment, the lubricant is stearic acid, palmitic acid, calcium hydroxide, talc, corn starch, sodium stearyl fumarate, sodium stearate, sodium benzoate, sodium lauryl sulfate, magnesium stearate, calcium stearate, zinc stearate, aluminum stearate, leucine, polyethylene glycol, glyceryl behenate, glyceryl monostearate, glyceryl palmitostearate, colloidal silicon dioxide, hydrogenated vegetable oil, hydrogenated castor oil, mineral oil, or waxes. In one embodiment, the lubricant is magnesium stearate or sodium stearyl fumarate. In one embodiment, the lubricant is sodium stearyl fumarate. In one embodiment, the lubricant provides a good workability of the formulation or composition blend during the compression phase (tableting). In one embodiment, lubricant can reduce surface irregularities of tablets. In one embodiment, a lubricant can be added to the composition and/or combination to reduce adhesion and ease the release of the product from the punch and dye.

In one embodiment, the formulation of the present disclosure comprises a sweetener. In one embodiment, the sweetener is selected from sugars or sugar alcohols. In one embodiment, the sweetener is selected from aspartame, ammonium glycyrrhizinate, sucralose, shaccarin sodium, sucrose, glucose, lactose, fructose, sorbitol, xylitol, or erythritol. In one embodiment, the sweetener is sucralose. In one embodiment, the sweetener is added to adjust the taste of the solubilized tablet.

In one embodiment, the formulation of the present disclosure comprises a binder. In one embodiment, the binder is selected from celluloses, cellulose ethers, cellulose esters, tricalcium phosphate, povidone (e.g., KOLLIDON, PLASDONE), copovidone, pregelatinized starch, dextrin, gelatin, pregelatinized starch, maltodextrin, starch, zein, acacia, alginic acid, carbomers (e.g., carbopol), cross-linked polyacrylates, polymethacrylates, gum tragacanth, guar gum, hydrogenated vegetable oil, magnesium aluminum silicate, liquid glucose, or sodium alginate. In one embodiment, the binder is selected from methylcellulose, carboxymethylcellulose, carboxymethylcellulose sodium, hydroxypropyl cellulose (e.g., KLUCEL), ethylcellulose, hydroxypropyl methylcellulose (e.g., METHOCEL), or hydroxyethyl cellulose. In one embodiment, the binder is hydroxypropyl methylcellulose (HPMC). In one embodiment, the binder helps to bind the carglumic acid and other excipient together after compression into a tablet or a dosage form.

In one embodiment, the formulation of the present disclosure comprises a binder known as Methocel E5®.

In one embodiment, the formulation of the present disclosure comprises a wetting agent. In one embodiment, the wetting agent is selected from sucrose palmitate, polyethylene glycol-polypropylene glycol copolymer, metal alkyl sulfate, sodium lauryl sulfate, polyoxyethylene sorbitan fatty acid ester, polyoxyethylene alkyl ether, polyethylene glycol, polyoxyethylene castor oil derivatives, docusate sodium, quaternary ammonium amine compounds, sugar esters of fatty acids, polyethoxylated fatty acid esters, glycerides of fatty acids, and polyglycolized glycerides. In one embodiment, the wetting agent is polyethylene glycol or sucrose palmitate. In one embodiment, the wetting agent is sucrose palmitate. In one embodiment, the wetting agent facilitates the wettability of carglumic acid.

In one embodiment, the formulation of the present disclosure comprises a glidant. In one embodiment, the glidant is selected from powdered cellulose, colloidal silicon dioxide, calcium silicate, magnesium trisilicate, talc, starch, corn starch, tribasic calcium phosphate, or a combination thereof. In one embodiment, the glidant is colloidal silicon dioxide. In one embodiment, the glidant is an anhydrous colloidal silicon dioxide. In one embodiment, the glidant improves flowability of the powder or non-compacted solid composition during tableting. In one embodiment, improving flowability increases the accuracy of dosing.

The pharmaceutical formulation of the present disclosure can further comprise a pharmaceutically acceptable carrier. In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers can include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable can include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers can include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use can include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

The dissolution rate of a compacted solid pharmaceutical composition in the patient's stomach may be increased by the addition of a disintegrant to the composition and/or combination. Disintegrants can include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. The agents or compounds, or pharmaceutically acceptable salts or derivatives thereof, are administered orally, nasally, transdermally, pulmonary, inhalationally, buccally, sublingually, intraperintoneally, subcutaneously, intramuscularly, intravenously, rectally, intrapleurally, intrathecally, intraportally, and parenterally. Alternatively or in addition, the route of administration is local, e.g., topical, intra-tumor and peri-tumor. In some embodiments, the compound is administered orally.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as water, fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In other embodiments the compound of the present disclosure can be administered by the intravenous route. In further embodiments, the parenteral administration may be provided in a bolus or by infusion.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

Tablets of the Present Disclosure

In one embodiment, the formulation of the present disclosure is in the form of a tablet. In one embodiment, the tablet is soluble in water. In one embodiment, the tablet disintegrates in water.

In one embodiment, the tablet formulation of the present disclosure has a disintegration time of no more than 5 minutes. In one embodiment, the tablet formulation of the present disclosure has a disintegration time of no more than 3 minutes. In one embodiment, the tablet formulation of the present disclosure has a disintegration time of less than 3 minutes. In one embodiment, the tablet formulation of the present disclosure has a disintegration time of less than 3 minutes in water. That is, the tablet formulation of the present disclosure disintegrates in water in less than about 180 seconds, less than about 170 seconds, less than about 160 seconds, less than about 150 seconds, less than about 140 seconds, less than about 130 seconds, less than about 120 seconds, less than about 110 seconds, less than about 100 seconds, less than about 90 seconds, less than about 80 seconds, less than about 70 seconds, or less than about 60 seconds. In one embodiment, the disintegration time is less than or equal to 90 seconds.

In one embodiment, a tablet formulation of the present disclosure comprising about 200 mg carglumic acid or a pharmaceutically acceptable salt thereof has a dissolution profile characterized by at least 85% dissolution in pH 1.2 hydrochloric acid medium at 37° C.±0.5° C. in no more than 15 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 90% dissolution in pH 1.2 hydrochloric acid medium at 37° C.±0.5° C. in no more than 15 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 95% dissolution in pH 1.2 hydrochloric acid medium at 37° C.±0.5° C. in no more than 15 minutes as measured by high-performance liquid chromatography at 200 nm.

In one embodiment, a tablet formulation of the present disclosure comprising about 200 mg carglumic acid or a pharmaceutically acceptable salt thereof has a dissolution profile characterized by at least 85% dissolution in pH 1.2 hydrochloric acid medium at 37° C.±0.5° C. in no more than 10 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 90% dissolution in pH 1.2 hydrochloric acid medium at 37° C.±0.5° C. in no more than 10 minutes as measured by high-performance liquid chromatography at 200 nm.

In one embodiment, a tablet formulation of the present disclosure comprising about 200 mg carglumic acid or a pharmaceutically acceptable salt thereof has a dissolution profile characterized by at least 85% dissolution in pH 4.5 sodium acetate buffer at 37° C.±0.5° C. in no more than 15 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 90% dissolution in pH 4.5 sodium acetate buffer at 37° C.±0.5° C. in no more than 15 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 95% dissolution in pH 4.5 sodium acetate buffer at 37° C.±0.5° C. in no more than 15 minutes as measured by high-performance liquid chromatography at 200 nm.

In one embodiment, a tablet formulation of the present disclosure comprising about 200 mg carglumic acid or a pharmaceutically acceptable salt thereof has a dissolution profile characterized by at least 85% dissolution in pH 4.5 sodium acetate buffer at 37° C.±0.5° C. in no more than 10 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 90% dissolution in pH 4.5 sodium acetate buffer at 37° C.±0.5° C. in no more than 10 minutes as measured by high-performance liquid chromatography at 200 nm.

In one embodiment, a tablet formulation of the present disclosure comprising about 200 mg carglumic acid or a pharmaceutically acceptable salt thereof has a dissolution profile characterized by at least 85% dissolution in pH 6.8 potassium phosphate buffer at 37° C.±0.5° C. in no more than 15 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 90% dissolution in pH 6.8 potassium phosphate buffer at 37° C.±0.5° C. in no more than 15 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 95% dissolution in pH 6.8 potassium phosphate buffer at 37° C.±0.5° C. in no more than 15 minutes as measured by high-performance liquid chromatography at 200 nm.

In one embodiment, a tablet formulation of the present disclosure comprising about 200 mg carglumic acid or a pharmaceutically acceptable salt thereof has a dissolution profile characterized by at least 85% dissolution in pH 6.8 potassium phosphate buffer at 37° C.±0.5° C. in no more than 10 minutes as measured by high-performance liquid chromatography at 200 nm. In one embodiment, a tablet formulation of the present disclosure comprising about 200 mg carglumic acid or a pharmaceutically acceptable salt thereof has a dissolution profile characterized by at least 90% dissolution in pH 6.8 potassium phosphate buffer at 37° C.±0.5° C. in no more than 10 minutes as measured by high-performance liquid chromatography at 200 nm.

In one embodiment, a unit dose of the present disclosure comprises about 100 mg to about 1000 mg of carglumic acid or a pharmaceutically acceptable salt thereof. In one embodiment, the unit dose formulation of claim 45, comprising about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, or about 850 mg of carglumic acid or a pharmaceutically acceptable salt thereof. In one embodiment, the unit dose formulation comprises about 200 mg of carglumic acid or a pharmaceutically acceptable salt thereof. In one embodiment, the unit dose formulation comprises about 800 mg of carglumic acid or a pharmaceutically acceptable salt thereof.

In one embodiment, the tablet formulation of the present disclosure has the composition disclosed in Table B.

TABLE B
Composition of 200 mg and 800 mg carglumic acid tablets

| | 200 mg Tablet | 800 mg Tablet |
|---|---|---|
| Carglumic Acid | 200.00 mg | 800.00 mg |
| Tromethamine | 110.00 mg | 440.00 mg |
| Pharmaburst ® | 140.25 mg | 561.00 mg |
| Effersoda ® | 20.00 mg | 80.00 mg |
| Methocel E5 | 15.00 mg | 60.00 mg |
| Sucrose Palmitate | 5.00 mg | 20.00 mg |
| Sucralose | 0.75 mg | 3.00 mg |
| Sodium Stearyl Fumarate | 5.00 mg | 20.00 mg |
| Colloidal Silica Dioxide | 4.00 mg | 16.00 mg |

In one embodiment, a unit dose of the present disclosure comprising about 200 mg carglumic acid comprises about 90 mg to about 150 mg tromethamine. In one embodiment, a unit dose of the present disclosure comprising about 200 mg carglumic acid comprises about 90 mg to about 120 mg tromethamine. In one embodiment, a unit dose of the present disclosure comprising about 200 mg carglumic acid comprises about 100 mg to about 120 mg tromethamine. In one embodiment, a unit dose of the present disclosure comprising about 200 mg carglumic acid comprises about 105 mg to about 115 mg tromethamine. In one embodiment, a unit dose of the present disclosure comprising about 200 mg carglumic acid comprises about 80 mg, about 90 mg, about 100 mg, about 110 mg, about 120 mg, about 130 mg, about 140 mg, about 150 mg, or about 160 mg tromethamine. In one embodiment, a unit dose of the present disclosure comprising about 200 mg carglumic acid comprises about 90 mg, about 95 mg, about 100 mg, about 105 mg, about 110 mg, about 115 mg, about 120 mg, about 125 mg, or about 130 mg of tromethamine. In one embodiment, a unit dose of the present disclosure comprising about 200 mg carglumic acid comprises about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg of tromethamine. In one embodiment, a unit dose of the present disclosure comprising about 200 mg carglumic acid comprises about 110 mg of tromethamine.

In one embodiment, a unit dose of the present disclosure comprising about 800 mg carglumic acid comprises about 400 mg to about 500 mg of tromethamine. In one embodiment, a unit dose of the present disclosure comprising about 800 mg carglumic acid comprises about 420 mg to about 460 mg of tromethamine. In one embodiment, a unit dose of the present disclosure comprising about 800 mg carglumic acid comprises about 430 mg to about 450 mg of tromethamine. In one embodiment, a unit dose of the present disclosure comprising about 800 mg carglumic acid comprises about 435 mg to about 445 mg of tromethamine. In one embodiment, a unit dose of the present disclosure comprising about 800 mg carglumic acid comprises about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, or about 520 mg of tromethamine. In one embodiment, a unit dose of the present disclosure comprising about 800 mg carglumic acid comprises about 420 mg, about 425 mg, about 430 mg, about 435 mg, about 440 mg, about 445 mg, about 450 mg, about 455 mg, or about 460 mg of tromethamine. In one embodiment, a unit dose of the present disclosure comprising about 800 mg carglumic acid comprises about 435 mg, about 436 mg, about 437 mg, about 438 mg, about 439 mg, about 440 mg, about 441 mg, about 442 mg, about 443 mg, about 444 mg, or about 445 mg of tromethamine. In one embodiment, a unit dose of the present disclosure comprising about 800 mg carglumic acid comprises about 440 mg of tromethamine.

In one embodiment, a unit dose of the present disclosure comprising about 200 mg carglumic acid comprises about 100 mg to about 200 mg of a combined total of fillers. In one embodiment, a unit dose of the present disclosure comprising about 200 mg carglumic acid comprises about 120 mg to about 160 mg of a combined total of fillers. In one embodiment, a unit dose of the present disclosure comprising about 200 mg carglumic acid comprises about 130 mg to about 150 mg of a combined total of fillers. In one embodiment, a unit dose of the present disclosure comprising about 200 mg carglumic acid comprises about 135 mg to about 145 mg of a combined total of fillers. In one embodiment, a unit dose of the present disclosure comprising about 800 mg carglumic acid comprises about 500 mg to about 600 mg of a combined total of fillers. In one embodiment, a unit dose of the present disclosure comprising about 800 mg carglumic acid comprises about 540 mg to about 580 mg of a combined total of fillers. In one embodiment, a unit dose of the present disclosure comprising about 800 mg carglumic acid comprises about 550 mg to about 570 mg of a combined total of fillers. In one embodiment, a unit dose of the present disclosure comprising about 800 mg carglumic acid comprises about 555 mg to about 565 mg of a combined total of fillers.

In one embodiment, a unit dose of the present disclosure comprising about 200 mg carglumic acid comprises about 100 mg to about 200 mg of Pharmaburst®. In one embodiment, a unit dose of the present disclosure comprising about 200 mg carglumic acid comprises about 120 mg to about 160 mg of Pharmaburst®. In one embodiment, a unit dose of the present disclosure comprising about 200 mg carglumic acid comprises about 140 mg to about 170 mg of Pharmaburst®. In one embodiment, a unit dose of the present disclosure comprising about 200 mg carglumic acid comprises about 150 mg to about 160 mg of Pharmaburst®. In one embodiment, a unit dose of the present disclosure comprising about 200 mg carglumic acid comprises about 130 mg to about 150 mg of Pharmaburst®. In one embodiment, a unit dose of the present disclosure comprising about 200 mg carglumic acid comprises about 135 mg to about 145 mg of Pharmaburst®. In one embodiment, a unit dose of the present disclosure comprising about 200 mg carglumic acid comprises about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, or about 220 mg of Pharmaburst®. In one embodiment, a unit dose of the present disclosure comprising about 200 mg carglumic acid comprises about 135 mg, about 136 mg, about 137 mg, about 138 mg, about 139 mg, about 140 mg, about 141 mg, about 142 mg, about 143 mg, about 144 mg, or about 145 mg Pharmaburst®. In one embodiment, a unit dose of the present disclosure comprising about 200 mg carglumic acid comprises about 140.00 mg, about 140.25 mg, about 140.50 mg, about 140.75 mg, or about 141.00 mg Pharmaburst®. In one embodiment, a unit dose of the present disclosure comprising about 200 mg carglumic acid comprises about 140.25 mg Pharmaburst®.

In one embodiment, a unit dose of the present disclosure comprising about 800 mg carglumic acid comprises about 400 mg to about 800 mg of Pharmaburst®. In one embodiment, a unit dose of the present disclosure comprising about 800 mg carglumic acid comprises about 480 mg to about 640 mg of Pharmaburst®. In one embodiment, a unit dose of the present disclosure comprising about 800 mg carglumic acid comprises about 560 mg to about 680 mg of Pharmaburst®. In one embodiment, a unit dose of the present disclosure comprising about 800 mg carglumic acid comprises about 600 mg to about 640 mg of Pharmaburst®. In one embodiment, a unit dose of the present disclosure comprising about 800 mg carglumic acid comprises about 500 mg to about 600 mg Pharmaburst®. In one embodiment, a unit dose of the present disclosure comprising about 800 mg carglumic acid comprises about 540 mg to about 580 mg Pharmaburst®. In one embodiment, a unit dose of the present disclosure comprising about 800 mg carglumic acid comprises about 550 mg to about 570 mg Pharmaburst®. In one embodiment, a unit dose of the present disclosure comprising about 800 mg carglumic acid comprises about 555 mg to about 565 mg Pharmaburst®. In one embodiment, a unit dose of the present disclosure comprising about 800 mg carglumic acid comprises about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, or about 620 mg Pharmaburst®. In one embodiment, a unit dose of the present disclosure comprising about 800 mg carglumic acid comprises about 540 mg, about 545 mg, about 550 mg, about 555 mg, about 560 mg, about 565 mg, about 570 mg, about 575 mg, or about 580 mg Pharmaburst®. In one embodiment, a unit dose of the present disclosure comprising about 800 mg carglumic acid comprises about 555 mg, about 556 mg, about 557 mg, about 558 mg, about 559 mg, about 560 mg, about 561 mg, about 562 mg, about 563 mg, about 564 mg, or about 565 mg Pharmaburst®. In one embodiment, unit dose of the present disclosure comprising about 800 mg carglumic acid comprises about 561 mg of Pharmaburst®.

In one embodiment, the formulation of the present disclosure comprises less than 1% of Impurity 1. In one embodiment, the formulation of the present disclosure comprises less than 1%, less than 0.9%, less than 0.8%, less than 0.7%, less than 0.6%, less than 0.5%, less than 0.4%, less than 0.3%, less than 0.2%, or less than 0.1% of Impurity 1. In one embodiment, the formulation of the present disclosure comprises less than 0.5% of Impurity 1. In one embodiment, the formulation of the present disclosure comprises less than 0.2% of Impurity 1. In one embodiment, the formulation of the present disclosure comprises less than or equal to 0.1% of Impurity 1. In one embodiment, the formulation of the present disclosure comprises less than 0.1% of Impurity 1. In one embodiment, the formulation of the present disclosure comprises less than or equal to 0.10% of Impurity 1. In one embodiment, the formulation of the present disclosure comprises less than 0.10% of Impurity 1.

In one embodiment, the formulation of the present disclosure comprises less than 1% of Impurity 1 after the formulation is stored at 25° C. at 60% relative humidity (RH) for 3 months. In one embodiment, the formulation of the present disclosure comprises less than 1%, less than 0.9%, less than 0.8%, less than 0.7%, less than 0.6%, less than 0.5%, less than 0.4%, less than 0.3%, less than 0.2%, or less than 0.1% of Impurity 1 after the formulation is stored at 25° C. at 60% relative humidity (RH) for 3 months. In one embodiment, the formulation of the present disclosure comprises less than 0.5% of Impurity 1 after the formulation is stored at 25° C. at 60% relative humidity (RH) for 3 months. In one embodiment, the formulation of the present disclosure comprises less than 0.2% of Impurity 1 after the formulation is stored at 25° C. at 60% relative humidity (RH) for 3 months. In one embodiment, the formulation of the present disclosure comprises less than or equal to 0.1% of Impurity 1 after the formulation is stored at 25° C. at 60% relative humidity (RH) for 3 months. In one embodiment, the formulation of the present disclosure comprises less than 0.1% of Impurity 1 after the formulation is stored at 25° C. at 60% relative humidity (RH) for 3 months. In one embodiment, the formulation of the present disclosure comprises less than 0.10% of Impurity 1 after the formulation is stored at 25° C. at 60% relative humidity (RH) for 3 months.

In one embodiment, the formulation of the present disclosure comprises less than 1% of Impurity 1 after the formulation is stored at 30° C. at 65% RH for 3 months. In one embodiment, the formulation of the present disclosure comprises less than 1%, less than 0.9%, less than 0.8%, less than 0.7%, less than 0.6%, less than 0.5%, less than 0.4%, less than 0.3%, less than 0.2%, or less than 0.1% of Impurity 1 after the formulation is stored at 30° C. at 65% RH for 3 months. In one embodiment, the formulation of the present disclosure comprises less than 0.5% of Impurity 1 after the formulation is stored at 30° C. at 65% RH for 3 months. In one embodiment, the formulation of the present disclosure comprises less than 0.2% of Impurity 1 after the formulation is stored at 30° C. at 65% RH for 3 months. In one embodiment, the formulation of the present disclosure comprises less than or equal to 0.1% of Impurity 1 after the formulation is stored at 30° C. at 65% RH for 3 months. In one embodiment, the formulation of the present disclosure comprises less than 0.1% of impurity 1 after the formulation is stored at 30° C. at 65% RH for 3 months. In one embodiment, the formulation of the present disclosure comprises less than 0.10% of Impurity 1 after the formulation is stored at 30° C. at 65% RH for 3 months.

Therapeutic Use

In one embodiment, the formulation of the present disclosure is useful for treating hyperammonaemia. As used herein, “hyperammonemia,” “hyperammonemic,” or “excess ammonia” refer to increased concentrations of ammonia in the body.

Hyperammonemia is caused by decreased detoxification and/or increased production of ammonia. Decreased detoxification may result from urea cycle disorders (UCDs), such as argininosuccinic aciduria, arginase deficiency, carbamoylphosphate synthetase deficiency, citrullinemia, N-acetylglutamate synthetase deficiency, and ornithine transcarbamylase deficiency, or from bypass of the liver (e.g., open ductus hepaticus), and/or deficiencies in glutamine synthetase. See WO2016200614A2, which is hereby incorporated by reference in its entirety. Decreased detoxification may also result from liver disorders such as hepatic encephalopathy, acute liver failure, or chronic liver failure, or from neurodegenerative disorders such as Huntington's disease. Increased production of ammonia may result from infections, drugs, neurogenic bladder, and intestinal bacterial overgrowth. Other disorders and conditions associated with hyperammonemia include, but are not limited to, liver disorders such as hepatic encephalopathy, acute liver failure, or chronic liver failure, organic acid disorders, isovaleric aciduria, 3-methylcrotonylglycinuria, methylmalonic academia, propionic aciduria, fatty acid oxidation defects, carnitine cycle defects, carnitine deficiency, β-oxidation deficiency, lysinuric protein intolerance, pyrroline-5-carboxylate synthetase deficiency, pyruvate carboxylase deficiency, ornithine aminotransferase deficiency, carbonic anhydrase deficiency, hyperinsulinism-hyperammonemia syndrome, mitochondrial disorders, valproate therapy, asparaginase therapy, total parenteral nutrition, cystoscopy with glycine-containing solutions, post-lung/bone marrow transplantation, portosystemic shunting; urinary tract infections, ureter dilation, multiple myeloma, infection, neurogenic bladder, or intestinal bacterial growth, and chemotherapy.

In healthy subjects, plasma ammonia concentrations are typically less than about 50 pmol/L. See WO2016200614A2.

In one embodiment, the formulation of the present disclosure is administered to a subject in need thereof, to treat hyperammonaemia.

In one embodiment, the formulation of the present disclosure is administered to a subject in need thereof, to treat conditions associated with hyperammonaemia. In one embodiment, the conditions associated with hyperammonaemia is liver disorders, organic acid disorders, isovaleric aciduria, 3-methylcrotonylglycinuria, methylmalonic academia, propionic aciduria, fatty acid oxidation defects, carnitine cycle defects, carnitine deficiency, β-oxidation deficiency, lysinuric protein intolerance, pyrroline-5-carboxylate synthetase deficiency, pyruvate carboxylase deficiency, ornithine aminotransferase deficiency, carbonic anhydrase deficiency, hyperinsulinism-hyperammonemia syndrome, mitochondrial disorders, urinary tract infections, ureter dilation, multiple myeloma, infection, neurogenic bladder, or intestinal bacterial growth. In one embodiment, liver disorder is hepatic encephalopathy, acute liver failure, or chronic liver failure.

In one embodiment, the formulation of the present disclosure is administered to a subject in need thereof, to decrease the plasma ammonia concentrations in the subject. In one embodiment, the formulation of the present disclosure is administered to a subject in need thereof, to decrease the plasma ammonia concentrations in the subject to less than about 50 pmol/L.

In one embodiment, the formulation of the present disclosure is administered to a subject in need thereof, to restore normal plasma ammonia concentrations in the subject.

In one embodiment, the subject in any one of the methods disclosed herein is human. In one embodiment, the subject is selected from pediatric population. In one embodiment, the subject is 18 years of age or less.

In one embodiment, the subject has a plasma ammonia concentration of at least about 50 pmol/L. In one embodiment, the subject has a plasma ammonia concentration of at least about 50 pmol/L, at least about 60 pmol/L, at least about 70 pmol/L, at least about 80 pmol/L, at least about 90 pmol/L, at least about 100 pmol/L, at least about 110 pmol/L, at least about 120 pmol/L, at least about 130 pmol/L, at least about 140 pmol/L, at least about 150 pmol/L, at least about 160 pmol/L, at least about 170 pmol/L, at least about 180 pmol/L, at least about 190 pmol/L, or at least about 200 pmol/L.

The pharmaceutical formulation of the disclosure may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The pharmaceutical formulation of the present disclosure may be manufactured and/or administered in single or multiple unit dose forms.

Having now generally described the invention, the same will be more readily understood through reference to the following examples, which are provided by way of illustration and are not intended to be limiting of the present invention.

### EXAMPLES

### Example 1

The stability of various carglumic acid/excipient combinations was tested as follows: a binary mix of active substance and excipient were prepared at different ratios, depending on the amount of excipient to be used in the final product, and then placed in HDPE bottle with silica gel.

The active substance-excipient combination ratios are presented in Table 1. Carglumic acid alone was also stored under the same conditions.

TABLE 1
Active Substance-Excipient Combination Ratios

| | | | | 25° C./60% | | 25° C./60% | | 40° C./75% | | |
|---|---|---|---|---|---|---|---|---|---|---|
| | | Ratio | | RH | | RH | | RH | | 60° C. |
| | | by | | Open | | Closed | | Closed | | Closed |
| | Excipient | weight | T = 0 | bottles | | bottles | | bottles | | bottles |
| Carglumic | — | 1 | X | 7 | 15 | 15 | 30 | 15 | 30 | 1 day |
| Acid | | | | days | days | days | days | days | days | |
| | Tromethamine | 1:5 | X | 7 | 15 | 15 | 30 | 15 | 30 | 1 day |
| | | | | days | days | days | days | days | days | |
| | Pharmaburst ® | 1:5 | X | 7 | 15 | 15 | 30 | 15 | 30 | 1 day |
| | | | | days | days | days | days | days | days | |
| | Effersoda ® | 1:5 | X | 7 | 15 | 15 | 30 | 15 | 30 | 1 day |
| | | | | days | days | days | days | days | days | |
| | Methocel E5 | 1:5 | X | 7 | 15 | 15 | 30 | 15 | 30 | 1 day |
| | | | | days | days | days | days | days | days | |
| | Sucrose | 1:1 | X | 7 | 15 | 15 | 30 | 15 | 30 | 1 day |
| | Palmitate | | | days | days | days | days | days | days | |
| | Sodium Stearyl | 1:1 | X | 7 | 15 | 15 | 30 | 15 | 30 | 1 day |
| | Fumarate | | | days | days | days | days | days | days | |
| | Aerosil | 1:1 | X | 7 | 15 | 15 | 30 | 15 | 30 | 1 day |
| | | | | days | days | days | days | days | days | |
| | Sucralose | 1:1 | X | 7 | 15 | 15 | 30 | 15 | 30 | 1 day |
| | | | | days | days | days | days | days | days | |

Chromatographic purity have been tested for all the reported combinations and storage conditions. Chromatographic conditions and gradient profile are summarized in the Tables 2 and 3. Under these chromatographic conditions the retention time (Rt) of carglumic acid is about 6.6 min. The compound having relative retention time (RRT) of 1.93 minutes using the HPLC conditions of Tables 2 and 3 is known as “Impurity 1.”

TABLE 2
Chromatographic Condition

| | |
|---|---|
| Column | Develosil 5 μm RPAQUEOUS-AR 250 × 4.6 mm |
| | (Phenomenex Part. No. CHO-5690) Pre-column |
| | Gemini C18 |
| Column temperature | 25° C. |
| Mobile Phase A | KH₂PO₄ 50 mM pH 2.0 per H₃PO₄ 85% |
| Mobile Phase B | MeOH |
| Flow rate | 1.0 mL/min |
| Injection volume | 50 μL |
| Autosampler | 15° C. |
| temperature | |
| Detection wavelength | UV at 215 nm |
| Elution mode | Gradient (see Table 2) |
| Run Time | 40 minutes |

TABLE 3
Gradient Profile

| TIME (min) | Mobile phase A % | Mobile phase B % |
|---|---|---|
| 0 | 100 | 0 |
| 8 | 100 | 0 |
| 14 | 90 | 10 |
| 28 | 90 | 10 |
| 30 | 100 | 0 |
| 40 | 100 | 0 |

Results obtained for the different active substance-excipients combinations are detailed in the Tables 4-12.

TABLE 4
Carglumic Acid Chromatographic Purity under Various Conditions (%)

| | | | | | | | | 60° C. |
|---|---|---|---|---|---|---|---|---|
| | | 25° C./60% RH | | 25° C./60% RH | | 40° C./75% RH | | Closed |
| | | Open bottles | | Closed bottles | | Closed bottles | | bottles |
| | t = 0 | 7 days | 15 days | 15 days | 30 days | 15 days | 30 days | 1 day |
| | White or | White or | White or | White or | White or | White or | White or | White or |
| | almost | almost | almost | almost | almost | almost | almost | almost |
| | white | white | white | white | white | white | white | white |
| | powder | powder | powder | powder | powder | powder | powder | powder |
| Imp. 6 | 0.00 | 0.00 | 0.00 | 0.00 | 0.00 | 0.00 | 0.00 | 0.00 |
| RRT 1.20 | | | | | | | | |
| Imp. 1 | 0.02 | 0.03 | 0.04 | 0.01 | 0.03 | 0.03 | 0.03 | 0.03 |
| RRT 1.93 | | | | | | | | |
| Imp. 2 | 0.01 | 0.01 | 0.01 | 0.00 | 0.01 | 0.01 | 0.01 | 0.01 |
| RRT 2.21 | | | | | | | | |
| Imp. 5a | 0.00 | 0.02 | 0.00 | 0.01 | 0.01 | 0.01 | 0.01 | 0.01 |
| RRT 2.34 | | | | | | | | |
| Imp. 5b | 0.01 | 0.00 | 0.00 | 0.00 | 0.00 | 0.01 | 0.00 | 0.00 |
| RRT 2.77 | | | | | | | | |
| Max, single | 0.03 | 0.05 | 0.02 | 0.03 | 0.03 | 0.02 | 0.03 | 0.02 |
| unknown | | | | | | | | |
| impurity | | | | | | | | |
| Total | 0.07 | 0.18 | 0.11 | 0.08 | 0.08 | 0.12 | 0.11 | 0.10 |
| impurities | | | | | | | | |

The results for tromethamine (Trometamol) and carglumic acid are shown in Table 5. After 7 days in open bottles the powder turned into a clear melted material. Without bound to any theory, this hygroscopic effect seems to have a negative impact on carglumic acid stability. Indeed after two weeks some unknown peaks were found in HPLC chromatograph. Also temperature had an effect on compatibility between the two component tested as evident by the high levels of Impurity 1.

Without being bound by any theory, the co-presence of tromethamine and moisture (already present internally or taken from the external environment) may trigger a degradation of the carglumic acid.

TABLE 5
Carglumic Acid and Tromethamine Mixture Chromatographic
Purity under Various Conditions (%)

| | | | | | | | | 60° C. |
|---|---|---|---|---|---|---|---|---|
| | | | | 25° C./60% RH | | | | Closed |
| | | | | Closed bottles | | | | bottles |
| | t = 0 | 25° C./60% RH | | 15 days | 30 days | 40° C./75% RH | | 1 day |
| | White or | Open bottles | | White or | White or | Closed bottles | | White or |
| | almost | 7 days | 15 days | almost | almost | 15 days | 30 days | almost |
| | white | Melted | Melted | white | white | Melted | Melted | white |
| | powder | material | material | powder | powder | material | material | powder |
| Imp. 6 | 0.00 | 0.00 | 0.00 | 0.00 | 0.00 | 0.00 | 0.00 | 0.00 |
| RRT 1.20 | | | | | | | | |
| Imp. 1 | 0.01 | 0.01 | 0.02 | 0.00 | 0.00 | 0.03 | 0.20 | 0.12 |
| RRT 1.93 | | | | | | | | |
| Imp. 2 | 0.01 | 0.00 | 0.01 | 0.00 | 0.00 | 0.00 | 0.03 | 0.03 |
| RRT 2.21 | | | | | | | | |
| Imp. 5a | 0.00 | 0.01 | 0.01 | 0.01 | 0.02 | 0.02 | 0.01 | 0.01 |
| RRT 2.34 | | | | | | | | |
| Imp. 5b | 0.01 | 0.00 | 0.00 | 0.00 | 0.00 | 0.00 | 0.00 | 0.00 |
| RRT 2.77 | | | | | | | | |
| Max, single | 0.03 | 0.07 | 0.28 | 0.00 | 0.02 | 0.03 | 0.24 | 0.26 |
| unknown | | | | | | | | |
| impurity | | | | | | | | |
| Total | 0.07 | 0.13 | 0.53 | 0.01 | 0.06 | 0.13 | 0.94 | 0.56 |
| impurities | | | | | | | | |

The results for Carglumic Acid and Pharmaburst® are shown in Table 6.

TABLE 6
Carglumic Acid and Pharmaburst ® Mixture Chromatographic Purity under Various Conditions (%)

| | | | | | | | | 60° C. |
|---|---|---|---|---|---|---|---|---|
| | | 25° C./60% RH | | 25° C./60% RH | | 40° C./75% RH | | Closed |
| | | Open bottles | | Closed bottles | | Closed bottles | | bottles |
| | t = 0 | 7 days | 15 days | 15 days | 30 days | 15 days | 30 days | 1 day |
| | White or | White or | White or | White or | White or | White or | White or | White or |
| | almost | almost | almost | almost | almost | almost | almost | almost |
| | white | white | white | white | white | white | white | white |
| | powder | powder | powder | powder | powder | powder | powder | powder |
| Imp. 6 | 0.01 | 0.00 | 0.00 | 0.00 | 0.01 | 0.00 | 0.00 | 0.00 |
| RRT 1.20 | | | | | | | | |
| Imp. 1 | 0.02 | 0.02 | 0.04 | 0.02 | 0.03 | 0.03 | 0.04 | 0.03 |
| RRT 1.93 | | | | | | | | |
| Imp. 2 | 0.01 | 0.01 | 0.01 | 0.01 | 0.01 | 0.01 | 0.01 | 0.01 |
| RRT 2.21 | | | | | | | | |
| Imp. 5a | 0.02 | 0.01 | 0.01 | 0.01 | 0.01 | 0.01 | 0.01 | 0.02 |
| RRT 2.34 | | | | | | | | |
| Imp. 5b | 0.01 | 0.00 | 0.00 | 0.00 | 0.00 | 0.00 | 0.00 | 0.00 |
| RRT 2.77 | | | | | | | | |
| Max, single | 0.03 | 0.04 | 0.03 | 0.04 | 0.15 | 0.06 | 0.04 | 0.06 |
| unknown | | | | | | | | |
| impurity | | | | | | | | |
| Total | 0.10 | 0.13 | 0.15 | 0.15 | 0.30 | 0.18 | 0.16 | 0.24 |
| impurities | | | | | | | | |

The results for Carglumic acid and Effersoda® are shown in Table 7.

TABLE 7
Carglumic Acid and Effersoda ® Mixture Chromatographic Purity under Various Conditions (%)

| | | | | | | | | 60° C. |
|---|---|---|---|---|---|---|---|---|
| | | 25° C./60% RH | | 25° C./60% RH | | 40° C./75% RH | | Closed |
| | | Open bottles | | Closed bottles | | Closed bottles | | bottles |
| | t = 0 | 7 days | 15 days | 15 days | 30 days | 15 days | 30 days | 1 day |
| | White or | White or | White or | White or | White or | White or | White or | White or |
| | almost | almost | almost | almost | almost | almost | almost | almost |
| | white | white | white | white | white | white | white | white |
| | powder | powder | powder | powder | powder | powder | powder | powder |
| Imp. 6 | 0.00 | 0.00 | 0.00 | 0.00 | 0.00 | 0.00 | 0.00 | 0.00 |
| RRT 1.20 | | | | | | | | |
| Imp. 1 | 0.00 | 0.02 | 0.03 | 0.01 | 0.03 | 0.03 | 0.04 | 0.02 |
| RRT 1.93 | | | | | | | | |
| Imp. 2 | 0.00 | 0.00 | 0.01 | 0.00 | 0.01 | 0.01 | 0.01 | 0.01 |
| RRT 2.21 | | | | | | | | |
| Imp. 5a | 0.00 | 0.01 | 0.01 | 0.01 | 0.01 | 0.00 | 0.02 | 0.01 |
| RRT 2.34 | | | | | | | | |
| Imp. 5b | 0.01 | 0.00 | 0.00 | 0.00 | 0.00 | 0.00 | 0.00 | 0.00 |
| RRT 2.77 | | | | | | | | |
| Max, single | 0.02 | 0.04 | 0.03 | 0.05 | 0.03 | 0.03 | 0.03 | 0.03 |
| unknown | | | | | | | | |
| impurity | | | | | | | | |
| Total | 0.04 | 0.07 | 0.09 | 0.07 | 0.12 | 0.09 | 0.12 | 0.09 |
| impurities | | | | | | | | |

Hypromellose 2910 (Methocel E5) and carglumic acid combination (Table 8) showed numerous unidentified peaks in the purity chromatogram at time 0. However, these peaks were related to the Methocel E5, and thus, determined to be not related to an carglumic acid degradation.

TABLE 8
Carglumic Acid and Methocel E5 Mixture Chromatographic Purity under Various Conditions (%)

| | | | | | | | | 60° C. |
|---|---|---|---|---|---|---|---|---|
| | | 25° C./60% RH | | 25° C./60% RH | | 40° C./75% RH | | Closed |
| | | Open bottles | | Closed bottles | | Closed bottles | | bottles |
| | t = 0 | 7 days | 15 days | 15 days | 30 days | 15 days | 30 days | 1 day |
| | White or | White or | White or | White or | White or | White or | White or | White or |
| | almost | almost | almost | almost | almost | almost | almost | almost |
| | white | white | white | white | white | white | white | white |
| | powder | powder | powder | powder | powder | powder | powder | powder |
| Imp. 6 | 0.00 | 0.00 | 0.00 | 0.01 | 0.00 | 0.00 | 0.01 | 0.00 |
| RRT 1.20 | | | | | | | | |
| Imp. 1 | 0.02 | 0.02 | 0.04 | 0.02 | 0.03 | 0.03 | 0.04 | 0.04 |
| RRT 1.93 | | | | | | | | |
| Imp. 2 | 0.01 | 0.01 | 0.01 | 0.01 | 0.01 | 0.01 | 0.01 | 0.01 |
| RRT 2.21 | | | | | | | | |
| Imp. 5a | 0.00 | 0.01 | 0.01 | 0.01 | 0.02 | 0.02 | 0.02 | 0.01 |
| RRT 2.34 | | | | | | | | |
| Imp. 5b | 0.00 | 0.00 | 0.02 | 0.02 | 0.00 | 0.00 | 0.02 | 0.00 |
| RRT 2.77 | | | | | | | | |
| Max, single | 0.27 | 0.34 | 0.29 | 0.33 | 0.36 | 0.37 | 0.34 | 0.31 |
| unkknown | | | | | | | | |
| impurity | | | | | | | | |
| Total | 0.72 | 0.77 | 0.71 | 0.70 | 0.75 | 0.81 | 0.79 | 0.74 |
| impurities | | | | | | | | |

The results for carglumic acid and sucrose palmitate are shown in Table 9.

TABLE 9
Carglumic Acid and Sucrose Palmitate Mixture Chromatographic Purity under Various Conditions (%)

| | | | | | | | | 60° C. |
|---|---|---|---|---|---|---|---|---|
| | | 25° C./60% RH | | 25° C./60% RH | | 40° C./75% RH | | Closed |
| | | Open bottles | | Closed bottles | | Closed bottles | | bottles |
| | t = 0 | 7 days | 15 days | 15 days | 30 days | 15 days | 30 days | 1 day |
| | White or | White or | White or | White or | White or | White or | White or | White or |
| | almost | almost | almost | almost | almost | almost | almost | almost |
| | white | white | white | white | white | white | white | white |
| | powder | powder | powder | powder | powder | powder | powder | powder |
| Imp. 6 | 0.00 | 0.00 | 0.00 | 0.00 | 0.00 | 0.00 | 0.00 | 0.00 |
| RRT 1.20 | | | | | | | | |
| Imp. 1 | 0.03 | 0.03 | 0.04 | 0.02 | 0.03 | 0.03 | 0.04 | 0.04 |
| RRT 1.93 | | | | | | | | |
| Imp. 2 | 0.01 | 0.01 | 0.01 | 0.01 | 0.01 | 0.01 | 0.01 | 0.01 |
| RRT 2.21 | | | | | | | | |
| Imp. 5a | 0.00 | 0.01 | 0.01 | 0.01 | 0.02 | 0.03 | 0.01 | 0.02 |
| RRT 2.34 | | | | | | | | |
| Imp. 5b | 0.01 | 0.00 | 0.01 | 0.00 | 0.00 | 0.00 | 0.00 | 0.00 |
| RRT 2.77 | | | | | | | | |
| Max, single | 0.03 | 0.27 | 0.11 | 0.03 | 0.10 | 0.02 | 0.03 | 0.02 |
| unknown | | | | | | | | |
| impurity | | | | | | | | |
| Total | 0.09 | 0.41 | 0.28 | 0.20 | 0.20 | 0.12 | 0.13 | 0.13 |
| impurities | | | | | | | | |

Carglumic acid and sodium stearyl fumarate mixture (Table 10) showed a chromatographic peak quite close to Impurity 1. Due to the high concentration of the excipient, specificity between Impurity 1 and sodium stearyl fumarate was lost.

TABLE 10
Carglumic Acid and Sodium Stearyl Fumarate Mixture
Chromatographic Purity under Various Conditions (%)

| | | | | | | | | 60° C. |
|---|---|---|---|---|---|---|---|---|
| | | 25° C./60% RH | | 25° C./60% RH | | 40° C./75% RH | | Closed |
| | | Open bottles | | Closed bottles | | Closed bottles | | bottles |
| | t = 0 | 7 days | 15 days | 15 days | 30 days | 15 days | 30 days | 1 day |
| | White or | White or | White or | White or | White or | White or | White or | White or |
| | almost | almost | almost | almost | almost | almost | almost | almost |
| | white | white | white | white | white | white | white | white |
| | powder | powder | powder | powder | powder | powder | powder | powder |
| Imp. 6 | 0.00 | 0.00 | 0.00 | 0.00 | 0.01 | 0.00 | 0.00 | 0.00 |
| RRT 1.20 | | | | | | | | |
| Imp. 1 | ND | ND | ND | ND | ND | ND | ND | ND |
| RRT 1.93 | | | | | | | | |
| Imp. 2 | 0.01 | 0.01 | 0.01 | 0.01 | 0.01 | 0.01 | 0.02 | 0.01 |
| RRT 2.21 | | | | | | | | |
| Imp. 5a | 0.00 | 0.01 | 0.02 | 0.02 | 0.01 | 0.01 | 0.01 | 0.01 |
| RRT 2.34 | | | | | | | | |
| Imp. 5b | 0.01 | 0.00 | 0.01 | 0.00 | 0.00 | 0.02 | 0.00 | 0.00 |
| RRT 2.77 | | | | | | | | |
| Max, single | 0.03 | 0.07 | 0.03 | 0.03 | 0.02 | 0.04 | 0.03 | 0.13 |
| unknown | | | | | | | | |
| impurity | | | | | | | | |
| Total | 0.10 | 0.21 | 0.20 | 0.08 | 0.08 | 0.19 | 0.09 | 0.40 |
| impurities | | | | | | | | |

Silica colloidal anhydrous (Aerosil) was fully compatible with the carglumic acid (Table 11). No unknown peaks were detected via HPLC.

TABLE 11
Carglumic Acid and Aerosil Mixture Chromatographic Purity under Various Conditions (%)

| | | | | | | | | 60° C. |
|---|---|---|---|---|---|---|---|---|
| | | 25° C./60% RH | | 25° C./60% RH | | 40° C./75% RH | | Closed |
| | | Open bottles | | Closed bottles | | Closed bottles | | bottles |
| | t = 0 | 7 days | 15 days | 15 days | 30 days | 15 days | 30 days | 1 day |
| | White or | White or | White or | White or | White or | White or | White or | White or |
| | almost | almost | almost | almost | almost | almost | almost | almost |
| | white | white | white | white | white | white | white | white |
| | powder | powder | powder | powder | powder | powder | powder | powder |
| Imp. 6 | 0.00 | 0.00 | 0.00 | 0.00 | 0.00 | 0.00 | 0.00 | 0.00 |
| RRT 1.20 | | | | | | | | |
| Imp. 1 | 0.02 | 0.03 | 0.05 | 0.02 | 0.04 | 0.04 | 0.04 | 0.04 |
| RRT 1.93 | | | | | | | | |
| Imp. 2 | 0.01 | 0.01 | 0.02 | 0.01 | 0.01 | 0.01 | 0.02 | 0.01 |
| RRT 2.21 | | | | | | | | |
| Imp. 5a | 0.00 | 0.02 | 0.01 | 0.01 | 0.02 | 0.01 | 0.02 | 0.01 |
| RRT 2.34 | | | | | | | | |
| Imp. 5b | 0.01 | 0.00 | 0.01 | 0.00 | 0.00 | 0.00 | 0.00 | 0.00 |
| RRT 2.77 | | | | | | | | |
| Max, single | 0.03 | 0.02 | 0.02 | 0.04 | 0.03 | 0.03 | 0.02 | 0.02 |
| unknown | | | | | | | | |
| impurity | | | | | | | | |
| Total | 0.08 | 0.12 | 0.14 | 0.12 | 0.13 | 0.11 | 0.11 | 0.11 |
| impurities | | | | | | | | |

Carglumic acid and sucralose mixture (Table 12) appeared to have triggered a darkening effect once stored at higher temperatures.

TABLE 12
Carglumic Acid and Sucralose Mixture Chromatographic Purity under Various Conditions (%)

| | | 25° C./60% RH | | 25° C./60% RH | | | | |
|---|---|---|---|---|---|---|---|---|
| | | Open bottles | | Closed bottles | | | | 60° C. |
| | t = 0 | 7 days | 15 days | 15 days | 30 days | 40° C./75% RH | | Closed |
| | White or | White or | White or | White or | White or | Closed bottles | | bottles |
| | almost | almost | almost | almost | almost | 15 days | 30 days | 1 day |
| | white | white | white | white | white | Black | Black | Black |
| | powder | powder | powder | powder | powder | spots | spots | powder |
| Imp. 6 | 0.00 | 0.00 | 0.00 | 0.00 | 0.00 | 0.00 | 0.00 | 0.00 |
| RRT 1.20 | | | | | | | | |
| Imp. 1 | 0.03 | 0.03 | 0.05 | 0.03 | 0.03 | 0.04 | 0.06 | 0.05 |
| RRT 1.93 | | | | | | | | |
| Imp. 2 | 0.01 | 0.01 | 0.01 | 0.01 | 0.01 | 0.01 | 0.02 | 0.01 |
| RRT 2.21 | | | | | | | | |
| Imp. 5a | 0.00 | 0.01 | 0.01 | 0.01 | 0.01 | 0.01 | 0.01 | 0.01 |
| RRT 2.34 | | | | | | | | |
| Imp. 5b | 0.01 | 0.00 | 0.00 | 0.00 | 0.00 | 0.00 | 0.00 | 0.01 |
| RRT 2.77 | | | | | | | | |
| Max, single | 0.03 | 0.03 | 0.03 | 0.02 | 0.02 | 0.02 | 0.41 | 0.03 |
| unknown | | | | | | | | |
| impurity | | | | | | | | |
| Total | 0.09 | 0.10 | 0.13 | 0.09 | 0.10 | 0.10 | 1.96 | 0.13 |
| impurities | | | | | | | | |

All excipient tested showed no significant negative effects at 25° C./60% RH conditions.

### Example 2

Prototypes A-E comprising Pharmaburst®, Effersoda®, and tromethamine as shown in Table 13 was prepared.

TABLE 13
Prototype Tablet Formulations (Prototype A-E)

| | A | B | C | D | E |
|---|---|---|---|---|---|
| Carglumic | 200 mg | 200 mg | 200 mg | 200 mg | 200 mg (sieved |
| Acid | | | | | through 250 μm) |
| Trometamol | | 96 mg | 96 mg | 96 mg | 96 mg |
| Pharmaburst ® | | 100 mg | 100 mg | 100 mg | 100 mg |
| Effersoda ® | | 20 mg | 40 mg | | |

Prototype A in water results in a high amount of undissolved powder. The bigger particles tend to fall down as sediment, and the thin particles tend to float.

Prototype B in water quickly disintegrated and a fine suspension remained.

Prototype C in water quickly disintegrated amd a fine suspension in water along with a thin layer of foam on the surface of solution remained.

Prototype D in water disintegrated slower than Prototypes B and C. A fine suspension in water remained.

Prototype E in water behaved similar to Prototype D. The finer particle size distribution (PSD) of the carglumic acid compared to Prototype D did not lead to a different behavior.

These disintegration/dissolution study with Prototypes A-E indicated that the PSD of the active substance does not affect neither its intrinsic solubility nor its kinetic of solubilization. Further, the presence of additional excipients improved the wettability of the carglumic acid, which in turns, led to an increased kinetic of solubilization.

Additional formulations were tested for manufacturability as shown in Table 14.

TABLE 14

| Component/Excipients (mg/tablet) | Trial 18 | Trial 17A | Trial 15B | Trial 16B | Trial 17C |
|---|---|---|---|---|---|
| Cargumic acid | 200 | 200 | 200 | 200 | 200 |
| Tromethamine (Buffer) | — | 95.5 | 127.3 | 63.7 | 95.5 |
| Effersoda ® (Effervescent Agent) | 10.0 | 10.0 | 20.0 | 20.0 | 20.0 |
| Pharmaburst ® (Filler) | 200 | 100 | 100 | 100 | 100 |
| Sucroester (Solubilizer/antifoam agent) | Absent | 4.16 | 4.56 | 4.00 | |
| Aerosil - colloidal silicon dioxide | 4.0 | 4.0 | 4.0 | 4.0 | 4.0 |
| (Glidant) | | | | | |
| Mg stearate (Lubricant) | 4.16 | 4.16 | 2.32 | 6.00 | 4.16 |
| Sucralose (Sweetener) | 0.40 | 0.40 | 0.48 | 0.40 | 0.40 |
| Total Weight | 414.1 | 418.3 | 458.6 | 398.0 | 414.1 |

Measured Critical Quality Attributes (CQAs)

| Appearance | | To be improved | | To be improved | | Good aspect | | To be improved | | To be improved | |
|---|---|---|---|---|---|---|---|---|---|---|---|
| | | (worn edges) | | (worn edges) | | of the tablet | | (worn edges) | | (worn edges) | |
| | | due to poor | | due to poor | | | | due to poor | | due to poor | |
| | | compactability | | compactability | | | | compactability | | compactability | |
| Hardness | | 21.6N | | 30.5N | | 58.4N | | 29.6N | | 27.7N | |
| | | X rel 14.5% | | X rel 7.9% | | X rel 6.4% | | X rel 10.3% | | X rel 13.0% | |
| Friability | | Not measurable | | 2.75% | | 1.15% | | Not measurable | | Not measurable | |
| Taste | | Sour taste | | Adequate | | Optimized | | Adequate | | Adequate | |
| Disintegration | <3 min | 0′37″ | | 0′53″ | | 1′42″ | | 0′22″ | | 0′56″ | |
| Dissolution | NLT 85% in 15 min | Complies | | Complies | | Complies | | Complies | | Complies | |
| Stability | | T = 0 | T = 30 d | T = 0 | T = 30 d | T = 0 | T = 30 d | T = 0 | T = 30 d | T = 0 | T = 30 d |
| summary | Δ Assay % | −1.7 | | −0.3 | | 1.5 | | −0.4 | | 1.2 | |
| 1 month at | % Glutamic Acid | 0.29 | 0.38 | 0.34 | 0.37 | 0.33 | 0.41 | 0.39 | 1.27 | 0.36 | 0.53 |
| 25° C./60% RH | % Total Known imp | 0.31 | 0.41 | 0.36 | 0.39 | 0.35 | 0.43 | 0.40 | 1.31 | 0.37 | 0.64 |
| in closed | % Total Unk imp | 0.04 | 0.04 | 0.10 | 0.04 | 0.06 | 0.03 | 0.07 | 0.04 | 0.10 | 0.02 |
| bottles | % Max Unk imp | 0.01 | 0.02 | 0.03 | 0.01 | 0.03 | 0.01 | 0.04 | 0.02 | 0.03 | 0.01 |

The formulation described in Table 15 was tested for stability.

TABLE 15

| | mg/tablet |
|---|---|
| Carglumic Acid | 200.00 |
| Tromethamine (Buffer) | 110.00 |
| Pharmaburst ® (Filler) | 155.25 |
| Aerosil (Glidant) | 4.00 |
| Effersoda ® (Effervescent Agent) | 20.00 |
| Sucrose Palmitate (Wetting Agent) | 5.00 |
| Sodium Stearyl Fumarate (Lubricant) | 5.00 |
| Sucralose (Sweetener) | 0.75 |
| Total Tablet weight | 500.00 |

The following manufacturing elements were further studied: shape of punches, coverage of punches surfaces with specific coating materials, and dry binder addition.

Shape of Punches: Several small scale 200 mg batches were manufactured by using different punches/tools. The tablets, having the composition of Table 15, were tested for hardness and breakability as summarized in Table 16.

TABLE 16
Summary of Hardness and Breakability
of Different 200 mg Strength Tablets

| Tablet Shape | | |
|---|---|---|
| Description | Hardness | Breakability |
| Oblong shape | The tablets did not | Not tested |
| 19 × 6.5 mm | reach acceptable | |
| 1 scoring line | values of hardness | |
| Round shape | 30N | do not pass USP test (6 portion |
| 12 mm diameter | | outside range) |
| 2 scoring lines | | [Tested for fracture in two |
| | | portions] |
| Round shape | 45N | Pass USP test. |
| 10 mm diameter | | The tablets are too small and the |
| 1 scoring lines | | breaking is difficult |
| Round shape | 50N | Not tested |
| 12 mm diameter | | |
| No scoring lines | | |
| Round shape | 50N | Pass USP test |
| 12 mm diameter | | |
| 1 scoring lines | | |

For the 800 mg Strength Tablets, it was determined that the use of 21 mm (diameter) round flat punches with one scoring line provided acceptable hardness and breakability. The 800 mg tablets used in this study had the composition in the ratio provided in Table 22, which was adjusted for 800 mg carglumic acid.

In order to adhere to the EP requirements for “Subdivision of Tablets” (<0478>) and USP requirements for the “Splitting Tablets with Functional Scoring” (USP <705>) the breakability analysis of tablets was carried out for both strengths:

EP requirements: the test requires a sample size of 30 tablets and the weight of one portion of each tablet has to fall within 85%-115% range of the mean of the weight of the tablet's portion calculated on the 30 tablets.

USP requirements: the test requires a sample size of 30 tablets and the weight of each portion of the tablet has to fall within 75%-125% range of the theoretical weight of the tablet's portion calculated on the real weight of the tablet.

For each strength the breakability test was performed with tablets at three different mean values of hardness (lower value, center point and upper value) studied in the Manufacturing. Validation Protocol. Results are summarized in Table 17. The 200 mg tablets used for this study had the composition of Table 22 and the 800 mg tablets had the composition in the ratio provided in Table 22, which was adjusted for 800 mg carglumic acid.

All tablets of the six types of tablet tested complied with the USP limits (all portions of tablet within 75%-125% range of the theoretical weight) and all tablets complied with the EP limits (all portions of tablet within 85%-115% of the average portion weight).

TABLE 17
Summary of Breakability Tests

| | Total | Theoretical | Weight | | USP % of | Weight | | USP % of |
|---|---|---|---|---|---|---|---|---|
| | weight | portion | Portion 1 | | Theoretical | Portion 2 | | Theoretical |
| | Avg (mg) | weight Avg | Avg (mg) | EP limits | weight Avg | Avg (mg) | *EP limits | weight Avg |
| Exp # | (n = 30) | (mg) (n = 30) | (n = 30) | portion 1 | (mg) (n = 30) | (n = 30) | portion 2 | (mg) (n = 30) |
| 1 | 200 mg tablet (50N) - Center Point of Tablet Hardness | | | | | | | |
| | 502.2 | 251.1 | 246.6 | 209.6-283.6 | 98.2 | 253.9 | 215.8-292.0 | 101.1 |
| 2 | 200 mg tablet (40N) - Lower Value of Tablet Hardness | | | | | | | |
| | 505.0 | 252.5 | 261.7 | 222.4-301.0 | 103.7 | 242.1 | 205.8-278.4 | 95.9 |
| 3 | 200 mg tablet (60N) - Upper Value of Tablet Hardness | | | | | | | |
| | 501.6 | 250.8 | 258.9 | 220.1-297.7 | 103.2 | 241.9 | 205.6-278.2 | 96.5 |
| 4 | 800 mg tablet (105N) - Center Point of Tablet Hardness | | | | | | | |
| | 2002.7 | 1001.3 | 1001.1 | 850.9-1151.3 | 100.0 | 999.5 | 849.6-1149.4 | 99.8 |
| 5 | 800 mg tablet (95N) - Lower Value of Tablet Hardness | | | | | | | |
| | 1948.1 | 974.0 | 989.2 | 840.8-1137.6 | 101.6 | 956.3 | 812.9-1100.1 | 98.2 |
| 6 | 800 mg tablet (120N) - Upper Value of Tablet Hardness | | | | | | | |
| | 2004.4 | 1002.2 | 1013.8 | 861.7-1165.9 | 101.2 | 987.0 | 839.0-1135.1 | 98.5 |

*Test was executed on both portion of the tablets

To further demonstrate that each portion of the tablet has an accurate fractional dose, tablet uniformity studies were conducted according to Ph. Eur chapter 2.9.40 and USP <905>. The summary of the uniformity tests is shown in Table 18. The 200 mg tablets used for this study had the composition of Table 22 and the 800 mg tablets had the composition in the ratio provided in Table 22, which was adjusted for 800 mg carglumic acid.

TABLE 18
Summary of Uniformity Tests (n = 30)

| | 200 mg tablet | 800 mg tablet |
|---|---|---|
| Median Assay (mg/cpr) | 196.13 | 791.37 |
| Standard Deviation Assay (mg/cpr) | 10.41 | 38.85 |
| Median Assay (%) | 98.07 | 98.92 |
| Standard Deviation Assay (%) | 5.21 | 4.86 |
| RSD % Assay (mg/cpr) | 5.31 | 4.91 |
| RSD % Assay (%) | 5.31 | 4.91 |
| Acceptance Value | 10.85 | 9.72 |

Acceptance value of less than 25.0 complies with the acceptance criteria. Thus, both strength tablets were determined to have acceptable content uniformity.

In addition, for both strengths, loss of mass percentage after splitting was calculated for all three hardness values studied as shown in Tables 19 and 20. The data obtained showed that the loss of mass percentage did not exceed the recommended value of 3%. The 200 mg tablets used for this study had the composition of Table 22 and the 800 mg tablets had the composition in the ratio provided in Table 22, which was adjusted for 800 mg carglumic acid.

TABLE 19
200 mg Strength Tablet Loss of Mass Percentage (n = 15)

| Hardness 50N | | | | Hardness 40N | | | | Hardness 60N | | | |
|---|---|---|---|---|---|---|---|---|---|---|---|
| Total | Portion | Portion | Loss of | Total | Portion | Portion | Loss of | Total | Portion | Portion | Loss of |
| (mg) | 1 (mg) | 2 (mg) | Mass % | (mg) | 1 (mg) | 2 (mg) | Mass % | (mg) | 1 (mg) | 2 (mg) | Mass % |
| 507.0 | 271.4 | 234.8 | 0.2 | 509.2 | 245.5 | 262.1 | 0.3 | 503.9 | 256.9 | 246.3 | 0.1 |
| 499.3 | 275.2 | 223.4 | 0.1 | 507.3 | 261.2 | 245.3 | 0.2 | 504.9 | 259.0 | 245.7 | 0.0 |
| 507.4 | 239.0 | 265.6 | 0.6 | 506.4 | 238.7 | 267.5 | 0.0 | 500.8 | 263.0 | 237.5 | 0.1 |
| 506.3 | 280.6 | 220.7 | 1.0 | 506.6 | 277.6 | 227.1 | 0.4 | 498.3 | 261.0 | 236.1 | 0.2 |
| 499.6 | 266.2 | 232.6 | 0.2 | 498.6 | 274.7 | 221.5 | 0.5 | 501.7 | 274.2 | 227.1 | 0.1 |
| 501.6 | 240.3 | 260.0 | 0.3 | 500.9 | 221.7 | 278.1 | 0.2 | 503.3 | 234.3 | 268.1 | 0.2 |
| 502.6 | 247.8 | 253.8 | 0.2 | 495.3 | 266.8 | 228.2 | 0.1 | 505.7 | 235.3 | 270.4 | 0.0 |
| 502.3 | 246.1 | 254.9 | 0.3 | 505.5 | 254.2 | 251.0 | 0.1 | 502.0 | 261.2 | 240.7 | 0.0 |
| 500.8 | 226.5 | 272.9 | 0.3 | 500.5 | 260.7 | 238.0 | 0.4 | 501.9 | 274.6 | 227.2 | 0.0 |
| 501.4 | 277.0 | 224.0 | 0.1 | 502.1 | 245.5 | 255.8 | 0.2 | 502.7 | 233.6 | 268.1 | 0.2 |
| 504.0 | 230.1 | 269.8 | 0.8 | 508.4 | 274.2 | 233.4 | 0.2 | 503.6 | 263.3 | 240.2 | 0.0 |
| 495.5 | 234.9 | 259.0 | 0.3 | 512.4 | 255.9 | 254.4 | 0.4 | 500.6 | 273.1 | 225.9 | 0.3 |
| 506.1 | 238.3 | 266.7 | 0.2 | 504.1 | 255.0 | 247.4 | 0.3 | 506.3 | 279.9 | 225.6 | 0.2 |
| 501.7 | 235.7 | 263.8 | 0.4 | 499.4 | 259.9 | 238.7 | 0.2 | 500.0 | 238.5 | 260.7 | 0.2 |
| 495.5 | 243.6 | 252.0 | 0.0 | 510.2 | 281.1 | 227.8 | 0.3 | 503.4 | 273.4 | 229.2 | 0.2 |

TABLE 20
800 mg Strength Tablet Loss of Mass Percentage (n = 15)

| Hardness 105N | | | | Hardness 95N | | | | Hardness 120N | | | |
|---|---|---|---|---|---|---|---|---|---|---|---|
| Total | Portion | Portion | Loss of | Total | Portion | Portion | Loss of | Total | Portion | Portion | Loss of |
| (mg) | 1 (mg) | 2 (mg) | Mass % | (mg) | 1 (mg) | 2 (mg) | Mass % | (mg) | 1 (mg) | 2 (mg) | Mass % |
| 2003.1 | 960.0 | 1041.7 | 0.1 | 1925.1 | 935.1 | 989.3 | 0.0 | 2021.9 | 1079.1 | 937.1 | 0.3 |
| 1982.9 | 988.1 | 992.5 | 0.1 | 1944.2 | 1016.6 | 925.9 | 0.1 | 2004.2 | 950.8 | 1050.9 | 0.1 |
| 2001.3 | 969.6 | 1030.4 | 0.1 | 1959.3 | 977.1 | 980.9 | 0.1 | 1986.9 | 1013.0 | 971.8 | 0.1 |
| 1973.3 | 998.5 | 971.8 | 0.2 | 1925.8 | 910.4 | 1013.9 | 0.1 | 1978.9 | 990.8 | 978.6 | 0.5 |
| 1981.9 | 1029.3 | 949.4 | 0.2 | 1907.6 | 941.3 | 964.7 | 0.1 | 1988.3 | 1061.8 | 924.9 | 0.1 |
| 2005.3 | 1104.4 | 897.1 | 0.2 | 1946.3 | 997.0 | 946.0 | 0.2 | 2027.6 | 1073.2 | 952.8 | 0.1 |
| 1975.7 | 952.0 | 1022.3 | 0.1 | 1954.4 | 1045.6 | 905.6 | 0.2 | 1981.4 | 1031.0 | 948.3 | 0.1 |
| 1990.5 | 964.1 | 1025.3 | 0.1 | 1929.2 | 1046.0 | 879.7 | 0.2 | 2004.0 | 1032.7 | 969.1 | 0.1 |
| 2050.1 | 1142.7 | 905.8 | 0.1 | 1923.8 | 1006.3 | 915.5 | 0.1 | 2014.1 | 1017.8 | 992.3 | 0.2 |
| 1985.1 | 909.0 | 1074.9 | 0.1 | 1969.1 | 893.3 | 1072.1 | 0.2 | 2014.8 | 925.3 | 1085.2 | 0.2 |
| 2064.3 | 1009.6 | 1051.3 | 0.2 | 1961.3 | 1120.8 | 837.3 | 0.2 | 1980.0 | 1003.0 | 974.3 | 0.1 |
| 1981.8 | 1018.1 | 962.9 | 0.0 | 1969.6 | 1032.9 | 932.8 | 0.2 | 1982.7 | 1046.2 | 934.0 | 0.1 |
| 2003.1 | 933.9 | 1066.7 | 0.1 | 1939.5 | 891.4 | 1043.6 | 0.2 | 1985.1 | 980.9 | 1002.3 | 0.1 |
| 1986.3 | 945.6 | 1039.2 | 0.1 | 1954.6 | 1062.2 | 888.8 | 0.2 | 1994.5 | 1040.5 | 952.1 | 0.1 |
| 1982.7 | 1002.6 | 979.1 | 0.1 | 1933.3 | 1030.3 | 901.1 | 0.1 | 1996.2 | 1017.8 | 976.9 | 0.1 |

To address sticking problem during tableting process, change in the amount of lubricant and addition of dry binders were considered. When the amount of sodium stearyl fumarate (lubricant) was increased up to 2% (compared to 1% w/w), the compression of the powder appeared to be positively affected; however, an increase of the lubricant caused an increase of the capping incidence.

Next, two types of dry binders (PVP K30 and Methocel E5, a low viscosity HPMC grade) were tested and resulting tablets were compared in terms disintegration time, residual matters on dissolution, hardness and incidence of sticking and capping. Composition of both formulas are detailed in Table 21.

TABLE 21
Formulation with Dry Binders

| Formula 1 | mg/tablet | Formula 2 | mg/tablet |
|---|---|---|---|
| Carglumic Acid | 200.00 | Carglumic Acid | 200.00 |
| Tromethamine | 110.00 | Tromethamine | 110.00 |
| Pharmaburst ® | 140.25 | Pharmaburst ® | 140.25 |
| Aerosil | 4.00 | Aerosil | 4.00 |
| Effersoda ® | 20.00 | Effersoda ® | 20.00 |
| PVP K30 | 15.00 | Methocel E5 | 15.00 |
| Sucrose Palmitate | 5.00 | Sucrose Palmitate | 5.00 |
| Sodium Stearyl | 5.00 | Sodium Stearyl | 5.00 |
| Fumarate | | Fumarate | |
| Sucralose | 0.75 | Sucralose | 0.75 |
| TOTAL | 500.00 | TOTAL | 500.00 |

Formulation 1 of Table 21 showed several problems of sticking and capping during the tableting of the blend.

Formulation 2 of Table 21 showed better results in terms of disintegration time, low residual on dissolution, adequate hardness while the capping problem was decreased. Thus, Formulation 2 was selected as the final formulation of the tablet as shown in Table 22 for 200 mg and 800 mg tablets.

TABLE 22
Carglumic Acid Tablet Formulation

mg/tablet

| | | |
|---|---|---|
| Carglumic Acid | 200.00 | 800.00 |
| Tromethamine | 110.00 | 440.00 |
| Pharmaburst ® | 140.25 | 561.00 |
| Aerosil | 4.00 | 16.00 |
| Effersoda ® | 20.00 | 80.00 |
| Methocel E5 | 15.00 | 60.00 |
| Sucrose Palmitate | 5.00 | 20.00 |
| Sodium Stearyl Fumarate | 5.00 | 20.00 |
| Sucralose | 0.75 | 3.00 |
| TOTAL | 500.00 | 2000.00 |

### Example 3

Batch 1. Small scale (5 kg) batch of 200 mg strength tablets having the composition and ratio as described in Table 22 was prepared.

Blending Phase

The first blend contained carglumic acid, Methocel E5, Pharmaburst® and Effersoda®. These excipients were sieved through a 500 μm sieve and manually mixed for 3 minutes.

The second blend contained tromethamine, Aerosil, sucrose palmitate and sucralose sieved through a 710 μm sieve and manually mixed for 3 minutes.

The two blends were then manually mixed together for 2 minutes. Finally, stearyl fumarate was added and mixed for 1 min 30 secs.

Tableting Phase

The Tableting machine was equipped with four 12 mm diameter punches with one scoring line. The process was carried out at two different speeds of the machine, setting the compression forces in order to obtain tablets with the desired properties. The tablets obtained were tested for hardness and disintegration as shown in Table 23.

TABLE 23
Hardness and Disintegration Time of Tablets In Small Scale Batch

| Measured Hardness range | 80-90N |
|---|---|
| Disintegration time | 1′10″-1′30″ |

Batch 2. A second small scale batch of tablets were prepared but this batch was split in two portion so to produce both 200 mg and 800 mg strength tablets. The blending phase process was carried out as discussed above for Batch 1. For the tableting phase, the tableting machine was equipped with four 12 mm diameter punches with one scoring line for the 200 mg strength and with four 21 mm diameter punches with scoring line for the 800 mg strength. For each dosage, the tablets were obtained at three different level of hardness in order to identify the final settings for the tableting machine. Data related to the compression of both strength are detailed in Tables 24 and 25.

TABLE 24
Compression Data for 200 mg Strength Tablets

| Press | Compression | Hardness (measured, | Tablet | Disintegration |
|---|---|---|---|---|
| speed | Force | average) | Height | time in seconds |
| 10 rpm | 16.5 KN | 40N | n.a. | 40-45 |
| 10 rpm | 25.0 KN | 60N | 3.9 mm | 60-70 |
| 10 rpm | 20.0 KN | 50N | 4.0 mm | 40-55 |
| 15 rpm | 21.0 KN | 50N | 4.0 mm | n.a. |
| 20 rpm | 21.0 KN | 50N | n.a. | n.a. |

TABLE 25
Compression Data for 800 mg Strength Tablets

| | | | | Disinte- | |
|---|---|---|---|---|---|
| Press | Compression | | Tablet | gration | Friability |
| speed | Force | Hardness | Height | time | (w/w) |
| 10 rpm | 50.0 KN | 115-135N | n.a. | n.a | na |
| 10 rpm | 48.0 KN | 95-115N | 5.7 mm | 50 seconds | 1.10% |
| 11 rpm | 45.0 KN | 75-95N | n.a | na | na |

The studied variations of process parameters such as shown in Tables 24 and 25 were useful to investigate the relationship with quality attributes such as hardness, disintegration time and friability.

### Example 4. Dissolution of Carglumic Acid Tablets

The key objective of the dissolution study was to compare the dissolution of the Carbaglu® Market tablet (see Table A for composition) and the Carglumic Acid tablets having the composition of Table 22.

Instrumentation:

Agilent UV (UV-Vis8453) automated dissolution apparatus

Dissolution system Apparatus 2 paddle

Analytical balance

pH meter

filter 10 μm

Materials and Chemicals Used:

Reference substances—Carglumic Acid working standard
Reagents—Potassium phosphate monobasic, Sigma-Aldrich
Water, (MilliQ purification)
Hydrochloric acid 0.1N solution, Sigma-Aldrich
Potassium chloride, Sigma-Aldrich
Sodium Acetate, Sigma-Aldrich
Acetic acid, Sigma-Aldrich
Sodium hydroxide 0.2 M, Fluka reagent

Dissolution Test Conditions:

The methodology used to perform the comparative dissolution testing is: NerPharMa S.r.l. analytical method 218ADS01-0, in compliance with USP <711>. Twelve (12) units have been tested with sampling times of 5, 10, 15, 20, and 30 minutes. In addition to the reference dissolution medium (pH 6.8), dissolution was also tested in two other buffers (pH 1.2 and pH 4.5).

Apparatus Paddle (apparatus 2)
Rotation speed 100 rpm
Temperature of the bath 37±0.5° C.
Medium a) Hydrochloric medium pH 1.2¹
b) Acetate buffer solution pH 4.5²
c) Phosphate buffer solution pH 6.8³

### Volume 750 ml

Sample one tablet per vessel (n=12)
Sampling time 5, 10, 15, 20, 30 minutes
¹Hydrochloric acid medium pH 1.2: Place 50 ml of 0.2 M potassium chloride (14.91 g of potassium chloride dissolved in deionized water and diluted to 1000.0 ml with the same solvent) in a 200 ml volumetric flask, add 85 ml of the hydrochloric acid solution 0.2 M and add water to volume.
²Acetate buffer solution pH 4.5: Dissolve 2.99 g of sodium acetate R in 750 ml of deionized water. Add 14.0 ml of 2M acetic acid and dilute to 1000.0 ml with deionized water.
³Phosphate buffer solution pH 6.8: Place 50 ml of the monobasic potassium phosphate of 0.2 M potassium dihydrogen phosphate (27.22 g of potassium dihydrogen phosphate R in 1000.0 ml of deionized water) with 22.4 ml of 0.2 M sodium hydroxide and dilute to 200 ml with deionized water.

Standard Solution:

About 27 mg of Acid Carglumic working standard and about 41 mg of placebo was weighed accurately into a 100 ml volumetric flask. The appropriate dissolution medium was added to the fill line.

Sample Solution:

Each tablet was placed into the dissolution vessel containing 750 ml of dissolution medium. Take the sample after the relevant sampling time, and measure the UV absorbance.

Calculations:

The amount % of Carglumic acid dissolved for each test sample was calculated. The percentage label dissolved at time t, Dt:

$$\frac{A u}{A s} \times \frac{C s}{L C} \times V \times 100 = \% {D}_{t}$$

where:

Dt=Percent dissolved at time t

Au=absorbance of sample solution at time t

As=absorbance of standard solution

LC=label claim (200 mg)

Cst=concentration (mg/ml) of standard solution

V=Volume of dissolution medium (ml)

Acceptance Criteria:

Where more than 85% of the labelled amount is dissolved within 15 minutes, dissolution profiles may be accepted as similar without further mathematical evaluation. In case of a lower dissolution rate than 85% at 15 min., a mathematical evaluation of similarity (f₂-test) would be performed.

The dissolution test results for 200 mg strength tablets are shown in Tables 26-28.

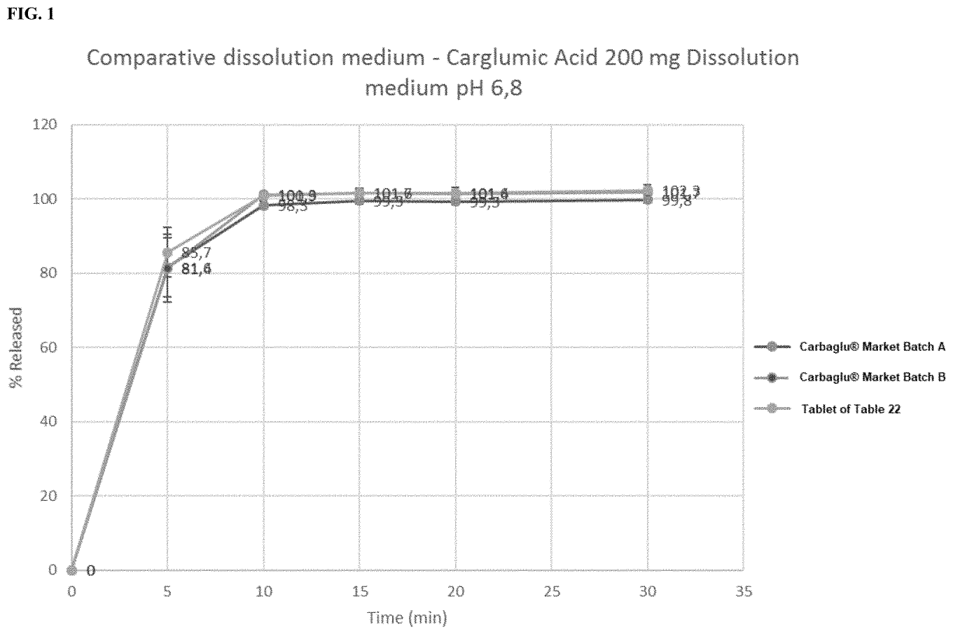

In all the tested conditions, the % released at 15 min was greater than 85% so, no further mathematical evaluation was needed. All the other acceptability assumptions (i.e. RSD of tablets at each specific time-point) were met. The dissolution profiles can be considered as similar in each tested condition. FIG. 1 shows Table 28 dissolution data in graphical form.

TABLE 26
Dissolution of 200 mg Strength Carglumic Acid Tablet in pH 1.2

| | Dosage Form | | | | | | | | | | | | | | |
|---|---|---|---|---|---|---|---|---|---|---|---|---|---|---|---|
| | Tablet of Table 22 | | | | | Carbaglu ® Market Batch A | | | | | Carbaglu ® Market Batch B | | | | |
| Time (min) | 5 | 10 | 15 | 20 | 30 | 5 | 10 | 15 | 20 | 30 | 5 | 10 | 15 | 20 | 30 |
| % Label | 81.2 | 97.1 | 98.6 | 98.7 | 99.6 | 77.7 | 96.6 | 97.3 | 97.7 | 98.5 | 85.9 | 100.2 | 102.8 | 103.7 | 105.3 |
| Claim | 72.4 | 99.2 | 100.6 | 101.1 | 102.5 | 68.4 | 98.8 | 100.3 | 100.4 | 100.9 | 75.0 | 100.4 | 102.0 | 102.2 | 102.7 |
| Released | 72.0 | 98.9 | 100.6 | 100.3 | 101.6 | 66.5 | 99.3 | 100.7 | 101.2 | 101.5 | 74.5 | 100.9 | 102.6 | 102.7 | 103.0 |
| | 85.3 | 99.1 | 100.3 | 100.4 | 101.4 | 82.4 | 98.0 | 101.1 | 99.9 | 99.9 | 89.9 | 100.6 | 101.8 | 101.9 | 102.1 |
| | 84.6 | 98.3 | 99.1 | 99.6 | 101.2 | 83.7 | 99.1 | 100.0 | 100.3 | 100.6 | 91.0 | 101.5 | 102.9 | 102.9 | 102.8 |
| | 89.3 | 98.4 | 99.1 | 99.6 | 100.5 | 89.2 | 99.0 | 100.3 | 100.5 | 102.3 | 96.2 | 102.9 | 103.9 | 103.8 | 103.9 |
| | 78.5 | 95.4 | 96.5 | 96.9 | 98.3 | 85.3 | 98.6 | 98.7 | 99.4 | 100.7 | 83.7 | 98.3 | 98.9 | 98.5 | 100.5 |
| | 57.3 | 98.0 | 99.4 | 99.8 | 101.3 | 72.4 | 96.6 | 96.9 | 97.9 | 98.9 | 72.1 | 99.9 | 101.3 | 101.0 | 102.7 |
| | 65.6 | 96.8 | 98.4 | 98.6 | 100.2 | 70.0 | 98.7 | 99.7 | 100.6 | 101.7 | 82.0 | 101.7 | 102.1 | 102.9 | 103.8 |
| | 80.4 | 96.4 | 97.9 | 97.8 | 99.1 | 87.9 | 99.3 | 99.3 | 100.4 | 101.3 | 88.4 | 100.6 | 101.0 | 101.1 | 102.5 |
| | 84.4 | 99.6 | 100.6 | 100.8 | 102.0 | 86.3 | 99.0 | 99.3 | 100.1 | 101.5 | 88.4 | 101.2 | 101.5 | 101.7 | 103.2 |
| | 87.8 | 97.2 | 98.2 | 98.4 | 99.6 | 90.0 | 97.8 | 98.0 | 99.0 | 99.7 | 93.2 | 101.5 | 102.0 | 102.2 | 103.4 |
| n | 12 | | | | | | | | | | | | | | |
| Average | 78.2 | 97.9 | 99.1 | 99.3 | 100.6 | 80.0 | 98.4 | 99.3 | 99.8 | 100.6 | 85.0 | 100.8 | 101.9 | 102.1 | 103.0 |
| % RDS | 12.3 | 1.29 | 1.3 | 1.3 | 1.3 | 10.8 | 1.0 | 1.3 | 1.1 | 1.2 | 9.1 | 1.1 | 1.2 | 1.4 | 1.1 |
| Min | 57.3 | 95.4 | 96.5 | 96.9 | 98.3 | 66.5 | 96.6 | 96.9 | 97.7 | 98.5 | 72.1 | 98.3 | 98.9 | 98.5 | 100.5 |
| Max | 89.3 | 99.6 | 100.6 | 101.1 | 102.5 | 90.0 | 99.3 | 101.1 | 101.2 | 102.3 | 96.2 | 102.9 | 103.9 | 103.8 | 105.3 |

TABLE 27
Dissolution of 200 mg Strength Carglumic Acid Tablet in pH 4.5

| | Dosage Form | | | | | | | | | | | | | | |
|---|---|---|---|---|---|---|---|---|---|---|---|---|---|---|---|
| | Tablet of Table 22 | | | | | Carbaglu ® Market Batch A | | | | | Carbaglu ® Market Batch B | | | | |
| Time (min) | 5 | 10 | 15 | 20 | 30 | 5 | 10 | 15 | 20 | 30 | 5 | 10 | 15 | 20 | 30 |
| % Label | 71.9 | 91.4 | 92.5 | 94.2 | 93.7 | 80.1 | 92.8 | 90.8 | 90.8 | 91.7 | 81.1 | 97.7 | 99.7 | 99.2 | 99.6 |
| Claim | 47.7 | 94.3 | 94.1 | 95.8 | 94.8 | 67.3 | 91.3 | 91.9 | 91.3 | 91.5 | 68.8 | 98.2 | 100.9 | 100.7 | 98.8 |
| Released | 61.4 | 93.6 | 94.2 | 96.1 | 96.9 | 72.9 | 92.2 | 91.9 | 90.6 | 93.2 | 70.6 | 97.3 | 99.8 | 99.6 | 97.5 |
| | 75.6 | 93.2 | 93.1 | 93.4 | 94.8 | 81.7 | 92.5 | 94.5 | 93.9 | 95.0 | 83.8 | 98.8 | 100.7 | 101.3 | 97.9 |
| | 75.7 | 94.4 | 93.4 | 94.9 | 95.5 | 80.9 | 93.2 | 92.6 | 92.8 | 95.7 | 83.8 | 96.9 | 98.3 | 99.5 | 100.5 |
| | 84.0 | 92.3 | 91.2 | 93.4 | 92.4 | 80.1 | 92.3 | 92.1 | 92.4 | 93.5 | 87.5 | 98.8 | 99.3 | 100.1 | 99.3 |
| | 78.3 | 99.8 | 99.0 | 99.3 | 101.4 | 77.0 | 90.8 | 90.2 | 91.3 | 91.9 | 79.3 | 94.3 | 93.5 | 95.7 | 94.9 |
| | 66.5 | 97.5 | 100.3 | 98.8 | 100.9 | 67.0 | 92.6 | 90.6 | 93.7 | 94.4 | 65.9 | 95.3 | 93.5 | 95.9 | 97.3 |
| | 67.0 | 99.3 | 99.9 | 100.2 | 102.6 | 66.5 | 92.0 | 92.1 | 93.6 | 91.1 | 63.2 | 93.8 | 93.3 | 94.9 | 95.5 |
| | 84.3 | 97.9 | 97.9 | 97.3 | 100.6 | 80.8 | 92.2 | 91.8 | 91.6 | 93.8 | 82.8 | 96.3 | 93.8 | 95.6 | 94.5 |
| | 83.5 | 97.2 | 98.5 | 97.7 | 100.4 | 77.5 | 92.4 | 92.1 | 91.1 | 91.9 | 82.4 | 95.8 | 95.2 | 96.1 | 96.1 |
| | 88.5 | 98.1 | 99.5 | 97.9 | 101.1 | 77.2 | 91.2 | 91.7 | 91.5 | 91.9 | 73.5 | 93.6 | 93.1 | 94.5 | 95.0 |
| n | 12 | | | | | | | | | | | | | | |
| Average | 73.7 | 95.8 | 96.1 | 96.6 | 97.9 | 75.8 | 92.1 | 91.9 | 92.1 | 93.0 | 76.9 | 96.4 | 96.8 | 97.8 | 97.2 |
| % RDS | 15.8 | 3.0 | 3.5 | 2.4 | 3.7 | 7.7 | 0.8 | 1.2 | 1.3 | 1.6 | 10.5 | 1.9 | 3.4 | 2.6 | 2.1 |
| Min | 47.7 | 91.4 | 91.2 | 93.4 | 93.7 | 66.5 | 90.8 | 90.2 | 90.6 | 91.1 | 63.2 | 93.6 | 93.1 | 94.5 | 94.5 |
| Max | 88.5 | 99.8 | 100.3 | 100.2 | 102.6 | 81.7 | 93.2 | 94.5 | 93.9 | 95.7 | 87.5 | 98.8 | 100.9 | 101.3 | 100.5 |

TABLE 28
Dissolution of 200 mg Strength Carglumic Acid Tablet in pH 6.8

| | Dosage Form | | | | | | | | | | | | | | |
|---|---|---|---|---|---|---|---|---|---|---|---|---|---|---|---|
| | Tablet of Table 22 | | | | | Carbaglu ® Market Batch A | | | | | Carbaglu ® Market Batch B | | | | |
| Time (min) | 5 | 10 | 15 | 20 | 30 | 5 | 10 | 15 | 20 | 30 | 5 | 10 | 15 | 20 | 30 |
| % Label | 86.6 | 100.5 | 101.8 | 102.0 | 102.7 | 82.9 | 95.8 | 98.1 | 97.5 | 98.3 | 86.0 | 101.5 | 101.9 | 102.0 | 102.0 |
| Claim | 76.3 | 102.0 | 103.1 | 103.4 | 104.2 | 71.8 | 98.5 | 100.1 | 99.7 | 100.3 | 74.1 | 101.6 | 102.3 | 102.4 | 102.3 |
| Released | 77.4 | 101.6 | 102.7 | 103.2 | 103.8 | 76.0 | 98.8 | 100.2 | 99.7 | 100.5 | 73.1 | 101.9 | 102.8 | 102.8 | 102.9 |
| | 89.1 | 102.2 | 103.3 | 103.7 | 104.2 | 85.8 | 98.1 | 99.3 | 98.9 | 99.6 | 82.5 | 100.5 | 101.0 | 101.0 | 101.0 |
| | 91.0 | 101.7 | 102.8 | 103.2 | 103.6 | 85.3 | 98.3 | 99.8 | 99.3 | 99.9 | 90.0 | 101.1 | 101.6 | 101.6 | 101.6 |
| | 87.3 | 98.9 | 99.7 | 100.2 | 100.7 | 85.6 | 98.2 | 99.6 | 99.4 | 99.8 | 88.6 | 101.7 | 102.5 | 102.2 | 102.2 |
| | 86.9 | 99.0 | 99.4 | 98.7 | 99.7 | 79.5 | 96.6 | 97.7 | 98.0 | 98.4 | 78.3 | 100.4 | 100.5 | 99.9 | 100.8 |
| | 81.0 | 102.0 | 102.5 | 102.0 | 103.0 | 77.7 | 99.6 | 100.4 | 100.5 | 100.7 | 71.9 | 102.2 | 102.4 | 102.1 | 102.7 |
| | 79.0 | 100.3 | 100.9 | 100.5 | 101.3 | 70.7 | 99.4 | 100.4 | 100.4 | 100.6 | 70.2 | 102.1 | 102.2 | 101.9 | 102.5 |
| | 92.4 | 101.4 | 101.9 | 101.3 | 102.1 | 84.5 | 98.4 | 99.0 | 99.1 | 99.3 | 84.3 | 101.3 | 101.2 | 100.8 | 101.4 |
| | 90.0 | 99.7 | 100.1 | 99.5 | 100.4 | 90.0 | 99.0 | 99.8 | 99.7 | 100.0 | 90.6 | 101.3 | 101.4 | 101.0 | 101.6 |
| | 90.8 | 101.3 | 101.9 | 101.3 | 102.4 | 89.4 | 99.2 | 99.8 | 99.9 | 100.1 | 86.9 | 99.4 | 99.3 | 99.0 | 99.7 |
| n | 12 | | | | | | | | | | | | | | |
| Average | 85.7 | 100.9 | 96.1 | 101.6 | 102.3 | 81.6 | 98.3 | 99.5 | 99.3 | 99.8 | 81.4 | 101.3 | 101.6 | 101.4 | 101.7 |
| % RDS | 6.7 | 1.2 | 3.5 | 1.6 | 1.5 | 7.9 | 1.1 | 0.9 | 0.9 | 0.8 | 9.2 | 0.8 | 1.0 | 1.1 | 0.9 |
| Min | 76.3 | 98.9 | 91.2 | 98.7 | 99.7 | 70.7 | 95.8 | 97.7 | 97.5 | 98.3 | 70.2 | 99.4 | 99.3 | 99.0 | 99.7 |
| Max | 92.4 | 102.2 | 100.3 | 103.7 | 104.2 | 90.0 | 99.6 | 100.4 | 100.5 | 100.7 | 90.6 | 102.2 | 102.8 | 102.8 | 102.9 |

### Example 5. In Vitro Dissolution of Carglumic Acid Tablets

The excipients used in Carbaglu® Market Tablets or the tablets of Table 22 are not intended to improve the absorption at the level of the stomach and the small intestine as none of the excipients are claimed as penetration/absorption enhancer; however, some of the excipients may contribute to improved solubility of carglumic acid through different physical effects:

Surfactants, such as sodium lauryl sulfate in Carbaglu® Market tablet and sucrose palmitate may improve the Drug Substance wettability, which in turn may lead to a slight increase of the overall solubility of the active substance. Both formulations include surfactants.

Buffering agents: Tromethamine may help carglumic acid solubilization through a shift of pH (shift to neutral pH, helping the acidic moiety to solubilize).

All other excipients, due to their nature as disintegrants, bulking agents/fillers, lubricant, glidants are not believed to alter the solubility of the active substance.

Concerning the process applied, the two formulations are manufactured through different procedures. Carbaglu® Market tablets are obtained via wet granulation followed by fluid bed drying and further blending and tableting phase while the carglumic acid tablets of Table 22 are manufactured by direct compression process through which the dry blend of the aforementioned excipients and the starting active ingredient are mixed and compressed without any other physical approach or modification.

The objective of this study was to investigate the in vitro dissolution behavior (through Compendial equipment) of Carbaglu® Market tablet and carglumic acid tablet of Table 22 upon dispersion/solubilization in water prior to oral administration.

A number of tablets were dispersed in 250 mL of water and the relevant gap of solubilized carglumic acid between formulations by starting from a highest dose (approx. 35 tablets in 250 mL of water) up to a lowest dose of 2.5 (200 mg) tablets in 250 mL of water, assuming, in both cases, 70 kg subject Body Weight (BW) as reference. These “in vitro” doses mimic the single administration of the drug considering a ter-in-die posology for the daily dose as indicated in the EU SmPC and US PI. Table 29 shows the single administration doses and the corresponding daily doses calculated for a simulated Body Weight of 70 kg.

TABLE 29
Single Administration Doses and Corresponding Daily Doses
Calculated for A Simulated Body Weight of 70 kg.

| | Number of tablets (single administration) | | | | | |
|---|---|---|---|---|---|---|
| | 2.5 | 5.0 | 7.5 | 13.0 | 18.5 | 35.0 |
| mg of Carglumic acid | 500.0 | 1000.0 | 1500.0 | 2600.0 | 3700.0 | 7000.0 |
| (single administration | | | | | | |
| amount) (200 mg/tablet) | | | | | | |
| Single Dose in mg/Kg | 7.1 | 14.3 | 21.4 | 37.1 | 52.9 | 100.0 |
| (posology) | | | | | | |
| Carglumic acid | 1.5 | 3.0 | 4.5 | 7.8 | 11.1 | 21.0 |
| (daily amount) in grams | | | | | | |
| Total Daily dose in mg/Kg | 21.4 | 42.9 | 64.3 | 111.4 | 158.6 | 300.0 |
| (posology) | | | | | | |

High Dose Simulation: 35 Tablets in 250 mL of Water

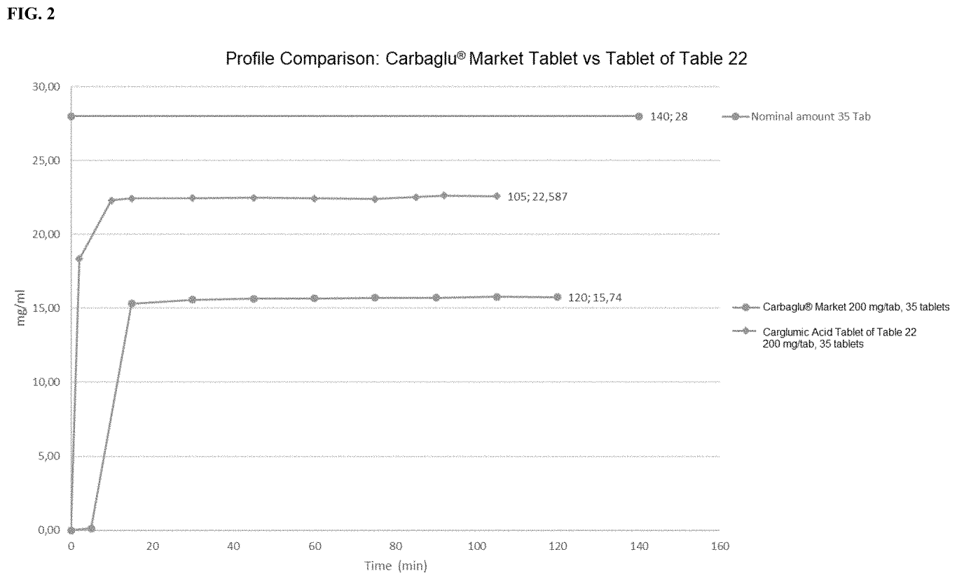

The solubilization/dispersion of 35 tablets (200 mg each) in 250 mL of water brought to a theoretical amount of carglumic acid equal to 28 mg/mL (represented by the plateau horizontal line in FIG. 2) which, in experimental testing, cannot be reached since it falls above the maximum solubility level of the active substance (at natural pH). The highest kinetic solubility is associated to the solubilization of the Table 22 carglumic acid tablets which reached, in a few minutes, a plateau of solubility of approx. 22.6 mg/mL whereas the Carbaglu® Market tablet allowed for carglumic acid solubilization of 15.7 mg/mL. The solubilization gap between the formulations (Carbaglu® Market and Table 22 tablet) of approximately 7 mg/mL is due to their composition differences. For both formulations, the differences to the nominal amount of 28 mg/mL consist of dispersed/suspended carglumic acid. See FIG. 2.

Lower Dose Simulation: 2.5 Tablets in 250 mL of Water

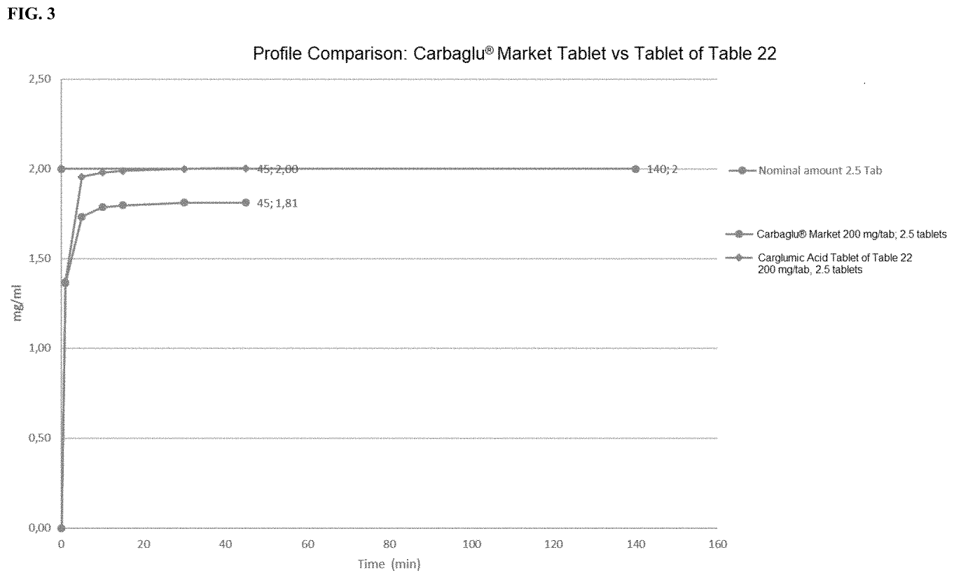

The solubilization/dispersion of 2.5 tablets in 250 mL of water would bring to a theoretical amount of carglumic acid of 2 mg/mL (represented by the plateau horizontal line in FIG. 3). The highest kinetic solubility is associated with the Table 22 carglumic acid tablets which reached in a few minutes a plateau of solubility of approx. 2 mg/mL (fitting the nominal expected) while the Carbaglu® Market tablet showed a value of 1.8 mg/mL. The between-formulation solubility gap was negligible (0.2 mg/mL). See FIG. 3.

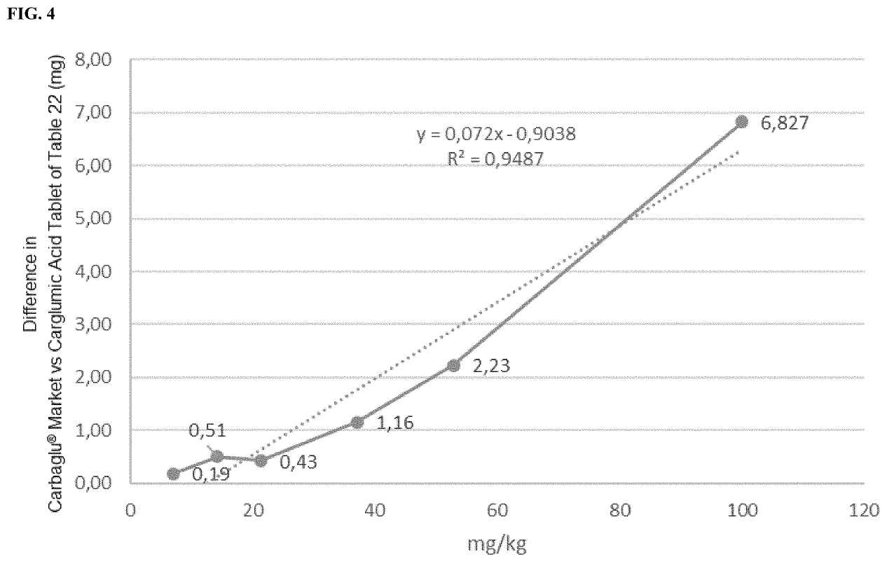

In addition to the two extremes dose regimens tested, several intermediate were simulated in vitro. It was clearly shown by the data reported in Table 30 that the solubility gap (mg/mL) between the carglumic acid tablet of Table 22 and Carbaglu® Market tablets increased more than proportionally to the dose regimen when solubilized/dispersed in 250 mL of water. A non-linear correlation between the mg/kg administered (amount of tablets solubilized in 250 mL of water), and the relevant solubility gap between formulations (indicated as mg/ml), is shown in FIG. 4.

TABLE 30
Correlation between the mg/kg administered and the
relevant solubility gap between formulations

| | Number of Tablets (single administration) | | | | | |
|---|---|---|---|---|---|---|
| | 2.5 | 5 | 7.5 | 13 | 18.5 | 35 |
| Difference in Carbaglu ® | 0.19 | 0.51 | 0.43 | 1.16 | 2.23 | 6.83 |
| Market vs the Table 22 | | | | | | |
| Tablet(mg/ml) | | | | | | |
| Difference in Carbaglu ® | 9.5 | 12.7 | 7.1 | 12.2 | 17.0 | 30.2 |
| Market vs the Table 22 Tablet | | | | | | |
| (%) | | | | | | |

FIG. 4 shows the solubility gap of the carglumic acid between Carbaglu® Market tablet and the formulations of Table 22 in relationship with the number of tablets solubilized in 250 mL of water. In FIG. 4, X axes reports the single administration doses in mg/kg (corresponding to a well-defined number of tablets in 250 mL of water), while Y axes indicates the differences (A) between the two formulations in terms of mg/ml of Carglumic acid solubilized in the medium. The overall slope of the dotted line (forced as linear regression) indicates the solubilizing power of the soluble formulation compared to the dispersible one: the steeper the line, the higher the difference among formulations.

As is shown up to a single dosage of 20-25 mg/kg a steady state of solubility gap is evident and the associated in vivo availability gap should be minimal or even indistinguishable from the physiological variability. A more than linear (likely exponential) increase of solubility gap appeared in the single dose range over 20-25 mg/kg up to 100 mg/kg.

In conclusion, the results of the tests clearly showed that the in vitro exponential progression of the in-between formulation solubility gap is moving towards higher single doses. Thus, without bound to any theory, it is reasonable to conclude that single doses up to 25-40 mg/kg should not trigger relevant differences of Cₘₐₓ ratio (Geometric Mean Ratio) of the PK indexes, while a statistically significant difference may be found when higher single doses are used, with a maximum Cₘₐₓ gap of approx. 30% at the highest extreme. This behavior observed in “in vitro” explains the “in vivo” dissolution data and related PK (pharmacokinetic) parameters, which showed an increase of the dissolved product from about 7% up to 30%, when an increased number of tablets were dispersed in water to allow the administration of high single doses in the smallest amount of water.

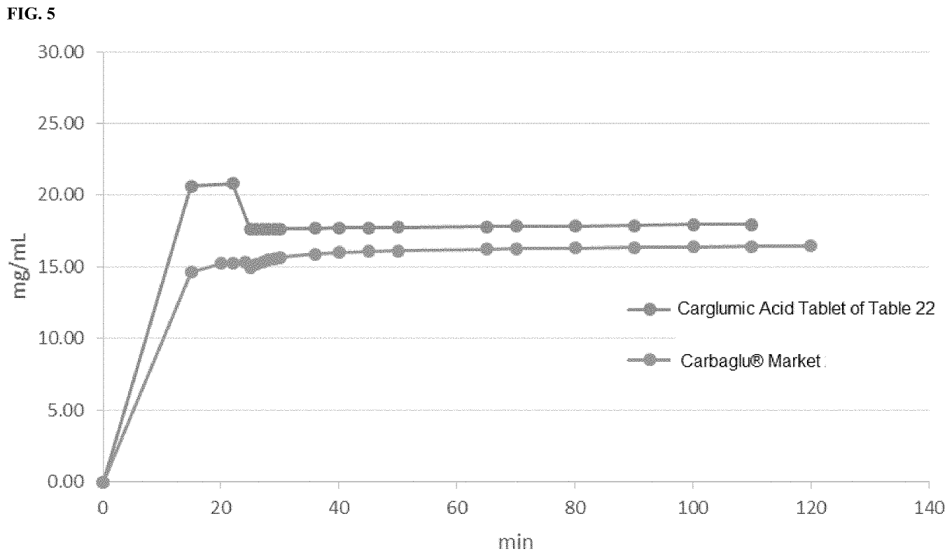

A further study in acid solution was performed to mimic the behavior of the solubilized product after administration. Both formulations were solubilized/dispersed in water (35 tablets—corresponding to 7000 mg Carglumic Acid—in 250 mL of water) and then a 250 mL of HCl 0.1N (simulating the gastric contact) were added after the complete dissolution. FIG. 5 shows the different behavior of the formulations in acidic solution.

Carbaglu® Market tablet reached a plateau of concentration in water after approximately 20 min and the addition of acid solution did not bring to any change in terms of carglumic acid solubility (FIG. 5). The carglumic acid tablet of Table 22 reached a higher concentration of Carglumic acid solubilized after 20 min which could be explained by, without bound to any theory, the presence of the tromethamine, acting as buffering/solubilizing element. Upon addition of acid solution, the solubilizing effect of the tromethamine is reduced and, as a consequence, a portion of the carglumic acid in solution re-precipitated in solid state.

### Example 6. Determination of Enantiomeric Purity of Carglumic Acid

Carglumic acid, a white powder or colorless crystals, is soluble in boiling water, slightly soluble in cold water, practically insoluble in organic solvents (cyclohexane, dichloromethane, ether). Carglumic acid has an asymmetric carbon atom (*) which may lead to isomerism; the Drug Substance exists in two enantiomeric forms, L- and D-Carglumic acid.

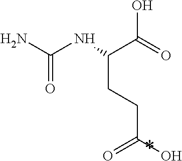

Synthetic process of L-carglumic acid used in the manufacturing of carglumic acid dosage form of the present invention starts from L-glutamic acid and none of the subsequent synthetic steps can lead to a change in the asymmetric carbon configuration. Nevertheless, the enantiomeric purity of the active substance is controlled at release and the specification limit has been fixed at <0.10% of D-carglumic acid.

Table 31 shows HPLC method used in determining enantiomeric purity of carglumic acid.

TABLE 31
HPLC Method

| | |
|---|---|
| Column | CHIROBIOTIC TAG, 1 = 250 × 4.6 mm 5 μm |
| Column temperature | 35° C. |
| Mobile Phase | 60% Acn/40% (KH₂PO₄ 25 mM pH 3.0 per |
| | H₃PO₄ 85%) |
| Flow rate | 0.5 mL/min |
| Injection volume | 10 μL |
| Autosampler temperature | 15° C. |
| Detection wavelength | UV at 200 nm |
| Run Time | 30 minutes |
| Concentration Sample | 5 mg/ml |
| solution | |
| Reference solution | 0.1% D-Carglumic Acid solution |

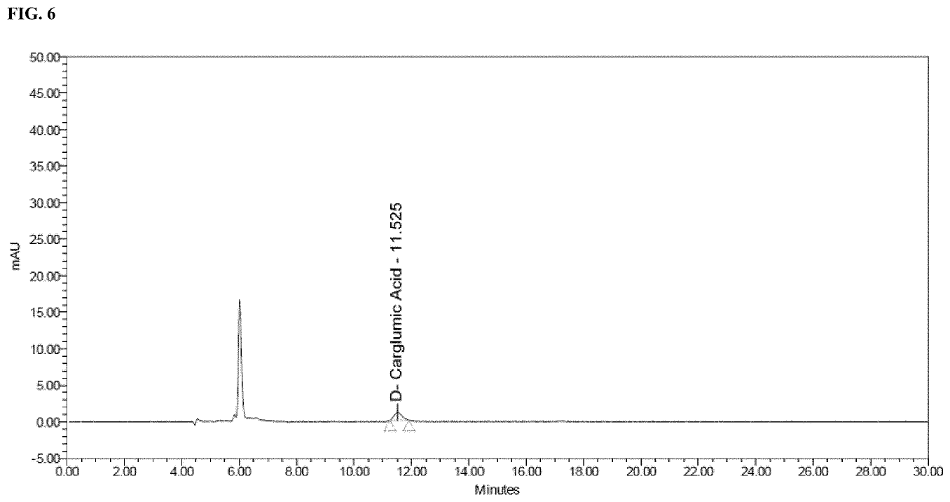

FIG. 6 shows chromatogram of a D-carglumic acid reference solution spiked at the specification limit of 0.1%. D-carglumic acid is observed at about 11.6 minutes using the HPLC method disclosed in Table 31, above.

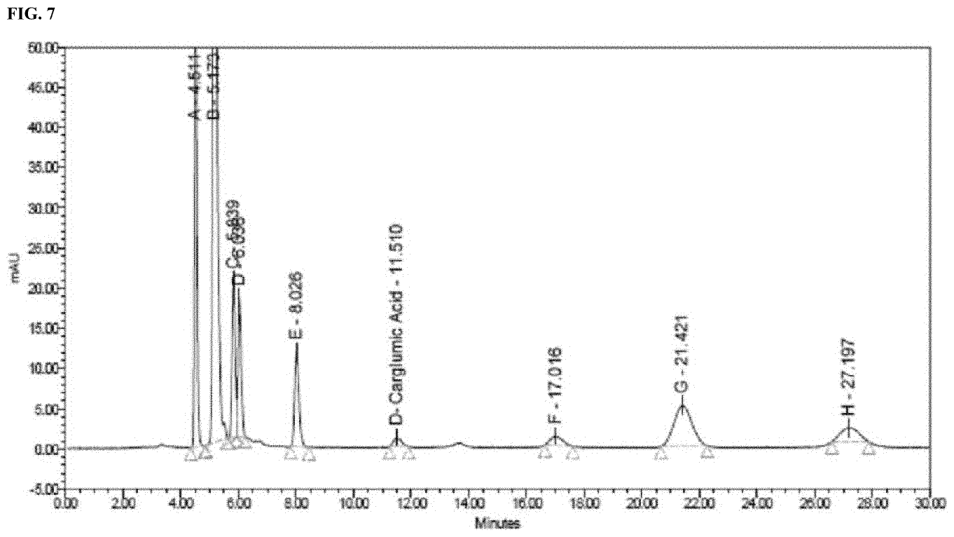

FIG. 7 shows chromatogram of a carglumic acid tablet having the composition of Table 22 which was spiked with 0.10% D-carglumic acid. FIG. 7 shows that this HPLC method is suitable to detect and quantify the presence of D-carglumic acid.

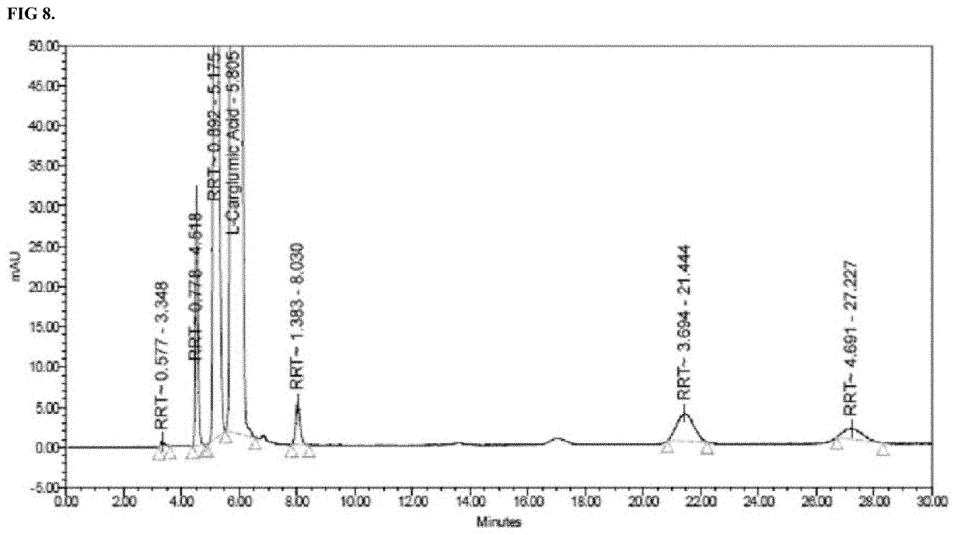

FIG. 8 shows chromatogram of 200 mg carglumic acid tablet having the composition in Table 22 with hardness value of 50 N. As shown in FIG. 8, D-carglumic acid was not detected in the tablet of the present invention. Same analysis was performed for tablets with hardness value of 40 N and 60 N. D-carglumic acid was observed in neither samples. Further, same experiment was conducted on 800 mg tablets having hardness values of 95 N, 105 N, and 120 N. No D-carglumic acid was observed in any of the tested samples.

### Example 7. Effects of Packaging

The effects of packaging material on stability was analyzed. 200 mg strength carglumic acid tablets having the composition of Table 15 were packaged in Alu/Alu Blister and in HDPE bottles with silica in the cap. Each were filled with 40 tablets. Both packaged products were tested under 25° C./60% RH, 30° C./65% RH, and 40° C./75% RH storage conditions up to 9 months. Tables 32-37 discloses the test results.

At 30° C./65% RH, specifically at months 6 and 9, it was already evident the more protective action of the HDPE bottle with silica compared to the Blister Alu/Alu was preferred. For example, see increase in Impurity 1 (which formation is mainly triggered by moisture intake) in Table 34 where the amount of Impurity 1 is above the limit of specification at months 6 and 9, while the same is well controlled when using bottles (compare with Table 35).

At 40° C./75% accelerated conditions, it was clearly evident the differences between Alu/Alu blisters and HDPE bottles with silica (compare Tables 36 and 37). Although at the last time point of 6 months, the stability in HDPE showed values greater than the specification related to Impurity 1 and disintegration time, the overall situation was dramatically worse in case of Alu/Alu blister where imputities above specifications appeared after 1 month.

TABLE 32
Carglumic Acid 200 mg Tablets packaged in Blister Alu/Alu, stored at 25° C./60% RH

| Parameter | Specification | 0 M | 1 M | 3 M | 6 M | 9 M |
|---|---|---|---|---|---|---|
| Friability | Report result | np | np | np | np | np |
| Uniformity of | According to Ph. Eur 2.9.40 | ≤15 | — | — | — | — |
| Dosage Unit | | | | | | |
| Assay | 90.0% to 105.0% of label | 96.8 | 98.7 | 100.9 | 101.6 | 101.8 |
| | claim | | | | | |
| Chromatographic | Imp. 6 RRT 1.20: ≤0.1% | 0.0 | 0.0 | 0.0 | 0.0 | 0.0 |
| Purity | | (0.00) | (0.00) | (0.00) | (0.00) | (0.00) |
| | Imp. 1 RRT 1.93: ≤0.1% | 0.0 | 0.0 | 0.0 | 0.1 | 0.1 |
| | | (0.02) | (0.02) | (0.03) | (0.06) | (0.09) |
| | Imp. 2 RRT 2.21: ≤0.1% | 0.0 | 0.0 | 0.0 | 0.0 | 0.0 |
| | | (0.01) | (0.01) | (0.00) | (0.01) | (0.01) |
| | Imp. 5a RRT 2.34: ≤0.1% | 0.0 | 0.0 | 0.0 | 0.0 | 0.0 |
| | | (0.01) | (0.01) | (0.01) | (0.01) | (0.01) |
| | Imp. 5b RRT 2.77: ≤0.1% | 0.0 | 0.0 | 0.0 | 0.0 | 0.0 |
| | | (0.00) | (0.00) | (0.00) | (0.00) | (0.01) |
| | Max, single unk. | 0.0 | 0.0 | 0.0 | 0.0 | 0.0 |
| | Impurity: ≤0.1% | (0.01) | (0.03) | (0.02) | (0.02) | (0.02) |
| | Total impurities ≤1.00% | 0.05 | 0.10 | 0.07 | 0.14 | 0.20 |
| Dissolution | ≥90% in 10 min | 112 | 104 | 98 | 101 | 104 |
| | | (109-113) | (103-106) | (96-99) | (99-102) | (100-106) |
| Loss on drying | Report result (%) | 1.93 | np | np | 1.35 | np |
| Disintegration | Report result (sec) | 1′17″ | 1′21″ | 2′07″ | 2′32″ | np |

np = not performed

TABLE 33
Carglumic Acid 200 mg Tablets packaged in HDPE Bottle, stored at 25° C./60% RH

| Parameter | Specification | 0 M | 1 M | 3 M | 6 M | 9 M |
|---|---|---|---|---|---|---|
| Friability | Report result | np | np | np | np | np |
| Uniformity of | According to Ph. Eur 2.9.40 | ≤15 | — | — | — | — |
| Dosage Unit | | | | | | |
| Assay | 90.0% to 105.0% of label | 96.8 | 97.5 | 99.8 | 101.6 | 101.0 |
| | claim | | | | | |
| Chromatographic | Imp. 6 RRT 1.20: ≤0.1% | 0.0 | 0.0 | 0.0 | 0.0 | 0.0 |
| Purity | | (0.00) | (0.00) | (0.00) | (0.00) | (0.00) |
| | Imp. 1 RRT 1.93: ≤0.1% | 0.0 | 0.0 | 0.0 | 0.0 | 0.0 |
| | | (0.02) | (0.02) | (0.01) | (0.03) | (0.04) |
| | Imp. 2 RRT 2.21: ≤0.1% | 0.0 | 0.0 | 0.0 | 0.0 | 0.0 |
| | | (0.01) | (0.01) | (0.00) | (0.01) | (0.02) |
| | Imp. 5a RRT 2.34: ≤0.1% | 0.0 | 0.0 | 0.0 | 0.0 | 0.0 |
| | | (0.01) | (0.01) | (0.01) | (0.01) | (0.00) |
| | Imp. 5b RRT 2.77: ≤0.1% | 0.0 | 0.0 | 0.0 | 0.0 | 0.0 |
| | | (0.00) | (0.00) | (0.00) | (0.00) | (0.01) |
| | Max, single unk. | 0.0 | 0.0 | 0.0 | 0.0 | 0.0 |
| | Impurity: ≤0.1% | (0.01) | (0.01) | (0.02) | (0.02) | (0.01) |
| | Total impurities ≤1.00% | 0.05 | 0.05 | 0.07 | 0.07 | 0.10 |
| Dissolution | ≥90% in 10 min | 112 | 101 | 98 | 100 | 101 |
| | | (109-113) | (100-103) | (97-100) | (99-101) | (99-106) |
| Loss on drying | Report result (%) | 1.93 | np | np | 1.13 | np |
| Disintegration | Report result (sec) | 1′17″ | 45″ | 53″ | 41″ | np |

np = not performed

TABLE 34
Carglumic Acid 200 mg Tablets packaged in Blister Alu/Alu, stored at 30° C./65% RH

| Parameter | Specification | 0 M | 1 M | 3 M | 6 M | 9 M |
|---|---|---|---|---|---|---|
| Friability | Report result | np | np | np | np | np |
| Uniformity of | According to Ph. Eur 2.9.40 | ≤15 | — | — | — | — |
| Dosage Unit | | | | | | |
| Assay | 90.0% to 105.0% of label | 96.8 | 97.0 | 99.7 | 101.2 | 99.6 |
| | claim | | | | | |
| Chromatographic | Imp. 6 RRT 1.20: ≤0.1% | 0.0 | 0.0 | 0.0 | 0.0 | 0.0 |
| Purity | | (0.00) | (0.00) | (0.00) | (0.00) | (0.00) |
| | Imp. 1 RRT 1.93: ≤0.1% | 0.0 | 0.0 | 0.1 | 0.4 | 0.2 |
| | | (0.02) | (0.03) | (0.07) | (0.38) | (0.24) |
| | Imp. 2 RRT 2.21: ≤0.1% | 0.0 | 0.0 | 0.0 | 0.0 | 0.0 |
| | | (0.01) | (0.01) | (0.01) | (0.03) | (0.02) |
| | Imp. 5a RRT 2.34: ≤0.1% | 0.0 | 0.0 | 0.0 | 0.0 | 0.0 |
| | | (0.01) | (0.02) | (0.01) | (0.01) | (0.01) |
| | Imp. 5b RRT 2.77: ≤0.1% | 0.0 | 0.0 | 0.0 | 0.0 | 0.0 |
| | | (0.00) | (0.00) | (0.00) | (0.01) | (0.02) |
| | Max, single unk. | 0.0 | 0.0 | 0.0 | 0.1 | 0.1 |
| | Impurity: ≤0.1% | (0.01) | (0.04) | (0.02) | (0.14) | (0.09) |
| | Total impurities ≤1.00% | 0.05 | 0.10 | 0.13 | 0.89 | 0.53 |
| Dissolution | ≥90% in 10 min | 112 | 105 | 96 | 100 | 101 |
| | | (109-113) | (103-106) | (93-97) | (99-101) | (100-102) |
| Loss on drying | Report result (%) | 1.93 | np | np | 1.32 | np |
| Disintegration | Report result (sec) | 1′17″ | np | 1′51″ | 3′33″ | np |

np = not performed

TABLE 35
Carglumic Acid 200 mg Tablets packaged in HDPE Bottle, stored at 30° C./65% RH

| Parameter | Specification | 0 M | 1 M | 3 M | 6 M | 9 M |
|---|---|---|---|---|---|---|
| Friability | Report result | np | np | np | np | np |
| Uniformity of | According to Ph. Eur 2.9.40 | ≤15 | — | — | — | — |
| Dosage Unit | | | | | | |
| Assay | 90.0% to 105.0% of label | 96.8 | 99.1 | 101.3 | 100.6 | 101.2 |
| | claim | | | | | |
| Chromatographic | Imp. 6 RRT 1.20: ≤0.1% | 0.0 | 0.0 | 0.0 | 0.0 | 0.0 |
| Purity | | (0.00) | (0.00) | (0.00) | (0.00) | (0.00) |
| | Imp. 1 RRT 1.93: ≤0.1% | 0.0 | 0.0 | 0.0 | 0.0 | 0.0 |
| | | (0.02) | (0.02) | (0.02) | (0.04) | (0.05) |
| | Imp. 2 RRT 2.21: ≤0.1% | 0.0 | 0.0 | 0.0 | 0.0 | 0.0 |
| | | (0.01) | (0.01) | (0.01) | (0.01) | (0.02) |
| | Imp. 5a RRT 2.34: ≤0.1% | 0.0 | 0.0 | 0.0 | 0.0 | 0.0 |
| | | (0.01) | (0.01) | (0.01) | (0.01) | (0.01) |
| | Imp. 5b RRT 2.77: ≤0.1% | 0.0 | 0.0 | 0.0 | 0.0 | 0.0 |
| | | (0.00) | (0.00) | (0.00) | (0.00) | (0.01) |
| | Max, single unk. | 0.0 | 0.0 | 0.0 | 0.0 | 0.0 |
| | Impurity: ≤0.1% | (0.01) | (0.04) | (0.02) | (0.02) | (0.01) |
| | Total impurities ≤1.00% | 0.05 | 0.10 | 0.07 | 0.09 | 0.11 |
| Dissolution | ≥90% in 10 min | 112 | 101 | 97 | 99 | 99 |
| | | (109-113) | (98-102) | (94-98) | (97-100) | (96-101) |
| Loss on drying | Report result (%) | 1.93 | np | np | 1.24 | np |
| Disintegration | Report result (sec) | 1′17″ | np | 1′05″ | 51″ | np |

np = not performed

TABLE 36
Carglumic Acid 200 mg Tablets packaged in
Blister Alu/Alu, stored at 40° C./75% RH

| Parameter | Specification | 0 M | 1 M | 3 M | 6 M |
|---|---|---|---|---|---|
| Friability | Report result | np | np | np | np |
| Uniformity of | According to Ph. Eur 2.9.40 | ≤15 | — | — | — |
| Dosage Unit | | | | | |
| Assay | 90.0% to 105.0% of label | 96.8 | 97.0 | 98.6 | 100.1 |
| | claim | | | | |
| Chromatographic | Imp. 6 RRT 1.20: ≤0.1% | 0.0 | 0.0 | 0.1 | 0.0 |
| Purity | | (0.00) | (0.00) | (0.07) | (0.00) |
| | Imp. 1 RRT 1.93: ≤0.1% | 0.0 | 0.2 | 0.6 | 1.3 |
| | | (0.02) | (0.18) | (0.62) | (1.31) |
| | Imp. 2 RRT 2.21: ≤0.1% | 0.0 | 0.0 | 0.1 | 0.1 |
| | | (0.01) | (0.02) | (0.05) | (0.10) |
| | Imp. 5a RRT 2.34: ≤0.1% | 0.0 | 0.0 | 0.0 | 0.0 |
| | | (0.01) | (0.01) | (0.01) | (0.02) |
| | Imp. 5b RRT 2.77: ≤0.1% | 0.0 | 0.0 | 0.0 | 0.0 |
| | | (0.00) | (0.00) | (0.01) | (0.03) |
| | Max, single unk. | 0.0 | 0.2 | 0.4 | 0.6 |
| | Impurity: ≤0.1% | (0.01) | (0.22) | (0.44) | (0.62) |
| | Total impurities ≤1.00% | 0.05 | 0.68 | 1.84 | 3.22 |
| Dissolution | ≥90% in 10 min | 112 | 96 | 83 | 85 |
| | | (109-113) | (92-99) | (80-86) | (83-88) |
| Loss on drying | Report result (%) | 1.93 | np | np | 1.31 |
| Disintegration | Report result (sec) | 1′17″ | 4′20″ | 4′58″ | 8′02″ |

np = not performed

TABLE 37
Carglumic Acid 200 mg Tablets packaged in HDPE Bottle, stored at 40° C./75% RH

| Parameter | Specification | 0 | 1 | 3 | 6 |
|---|---|---|---|---|---|
| Friability | Report result | np | np | np | np |
| Uniformity of | According to Ph. Eur 2.9.40 | ≤15 | — | — | — |
| Dosage Unit | | | | | |
| Assay | 90.0% to 105.0% of label | 96.8 | 99.5 | 99.5 | 100.2 |
| | claim | | | | |
| Chromatographic | Imp. 6 RRT 1.20: ≤0.1% | 0.0 | 0.0 | 0.0 | 0.0 |
| Purity | | (0.00) | (0.00) | (0.00) | (0.00) |
| | Imp. 1 RRT 1.93: ≤0.1% | 0.0 | 0.0 | 0.1 | 0.3 |
| | | (0.02) | (0.03) | (0.10) | (0.26) |
| | Imp. 2 RRT 2.21: ≤0.1% | 0.0 | 0.0 | 0.0 | 0.0 |
| | | (0.01) | (0.01) | (0.01) | (0.02) |
| | Imp. 5a RRT 2.34: ≤0.1% | 0.0 | 0.0 | 0.0 | 0.0 |
| | | (0.01) | (0.01) | (0.01) | (0.01) |
| | Imp. 5b RRT 2.77: ≤0.1% | 0.0 | 0.0 | 0.0 | 0.0 |
| | | (0.00) | (0.00) | (0.00) | (0.01) |
| | Max, single unk. | 0.0 | 0.0 | 0.0 | 0.0 |
| | Impurity: ≤0.1% | (0.01) | (0.03) | (0.02) | (0.07) |
| | Total impurities ≤1.00% | 0.05 | 0.13 | 0.23 | 0.54 |
| Dissolution | ≥90% in 10 min | 112 | 100 | 95 | 97 |
| | | (109-113) | (99-103) | (94-98) | (92-100) |
| Loss on drying | Report result (%) | 1.93 | np | np | 0.99 |
| Disintegration | Report result (sec) | 1′17″ | 1′25″ | 2′34″ | 3′56″ |

Next, the effects of number of tablets (having the composition of Table 22) in HDPE bottles were assessed under various storage conditions (40 tablets in one bottle vs 60 tablets in one bottle). Tables 38-45 shows the study results.

The out of specification at 3 months 40° C./75% RH for fill count of 40-tablet (Table 40) may be an outlier due to other unknown causes as the value of the Impurity 1 quickly grew up to more than 2%, which was not expected looking at the trend up to the 1st month (1M).

TABLE 38
Carglumic Acid 200 mg Tablets (40) packaged
in HDPE Bottle, stored at 5° C.

| Parameter | Specification | 0 M | 1 M |
|---|---|---|---|
| Hardness | Report result | np | np |
| Assay | 90.0% to 105.0% of label claim | 99.9 | 98.5 |
| Chromatographic | Imp. 6 RRT 1.20: ≤0.1% | ND | ND |
| Purity | Imp. 1 RRT 1.93: ≤0.1% | 0.03 | 0.03 |
| | Imp. 2 RRT 2.21: ≤0.1% | 0.01 | 0.01 |
| | Imp. 5a RRT 2.34: ≤0.1% | 0.01 | 0.01 |
| | Imp. 5b RRT 2.77: ≤0.1% | ND | ND |
| | Max, single unk. | 0.02 | 0.02 |
| | Impurity: ≤0.1% | | |
| | Total impurities: Report result | 0.1 | 0.1 |
| Dissolution | ≥85% in 15 min | 96 | np |
| | | (95-98) | |
| Loss on drying | Report result | 1.3 | np |
| Disintegration | Report result | <1 min | <1 min |

np = not performed;
ND = not detectable

TABLE 39
Carglumic Acid 200 mg Tablets (40) packaged
in HDPE Bottle, stored at 25° C./60% RH

| Parameter | Specification | 0 M | 1 M | 3 M |
|---|---|---|---|---|
| Hardness | Report result | np | np | np |
| Assay | 90.0% to 105.0% of label | 99.9 | 98.6 | 100.3 |
| | claim | | | |
| Chroma- | Imp. 6 RRT 1.20: ≤0.1% | ND | ND | ND |
| tographic | Imp. 1 RRT 1.93: ≤0.1% | 0.03 | 0.03 | 0.02 |
| Purity | Imp. 2 RRT 2.21: ≤0.1% | 0.01 | 0.01 | 0.01 |
| | Imp. 5a RRT 2.34: ≤0.1% | 0.01 | 0.01 | 0.00 |
| | Imp. 5b RRT 2.77: ≤0.1% | ND | ND | ND |
| | Max, single unk. | 0.02 | 0.02 | 0.01 |
| | Impurity: ≤0.1% | | | |
| | Total impurities: Report | 0.1 | 0.1 | 0.04 |
| | result | | | |
| Dissolution | ≥85% in 15 min | 96 | np | 100 |
| | | (95-98) | | (99-101) |
| Loss on drying | Report result | 1.3 | np | np |
| Disintegration | Report result | <1 min | 55″ | 1′02″ |

np = not performed;
ND = not detectable

TABLE 40
Carglumic Acid 200 mg Tablets (40) packaged
in HDPE Bottle, stored at 40° C./75% RH

| Parameter | Specification | 0 M | 1 M | 3 M |
|---|---|---|---|---|
| Hardness | Report result | np | np | np |
| Assay | 90.0% to 105.0% of label | 99.9 | 98.9 | 96.4 |
| | claim | | | |
| Chromatographic | Imp. 6 RRT 1.20: ≤0.1% | ND | ND | ND |
| Purity | Imp. 1 RRT 1.93: ≤0.1% | 0.03 | 0.06 | 2.06 |
| | Imp. 2 RRT 2.21: ≤0.1% | 0.01 | 0.01 | 0.29 |
| | Imp. 5a RRT 2.34: ≤0.1% | 0.01 | 0.01 | ND |
| | Imp. 5b RRT 2.77: ≤0.1% | ND | ND | 0.05 |
| | Max, single unk. | 0.02 | 0.02 | 0.67 |
| | Impurity: ≤0.1% | | | |
| | Total impurities: Report | 0.1 | 0.1 | 4.85 |
| | result | | | |
| Dissolution | ≥85% in 15 min | 96 | np | 88 |
| | | (95-98) | | (85-91) |
| Loss on drying | Report result | 1.3 | np | np |
| Disintegration | Report result | <1 min | 2′40″ | 7′00″ |

np = not performed;
ND = not detectable

TABLE 41
Carglumic Acid 200 mg Tablets (40) packaged
in HDPE Bottle, stored at 30° C./65% RH

| Parameter | Specification | 0 M | 3 M |
|---|---|---|---|
| Hardness | Report result | np | np |
| Assay | 90.0% to 105.0% of label claim | 99.9 | 98.4 |
| Chromatographic | Imp. 6 RRT 1.20: ≤0.1% | ND | ND |
| Purity | Imp. 1 RRT 1.93: ≤0.1% | 0.03 | 0.03 |
| | Imp. 2 RRT 2.21: ≤0.1% | 0.01 | 0.01 |
| | Imp. 5a RRT 2.34: ≤0.1% | 0.01 | ND |
| | Imp. 5b RRT 2.77: ≤0.1% | ND | ND |
| | Max, single unk. | 0.02 | ND |
| | Impurity: ≤0.1% | | |
| | Total impurities: Report result | 0.1 | 0.04 |
| Dissolution | ≥85% in 15 min | 96 | 100 |
| | | (95-98) | (99-101) |
| Loss on drying | Report result | 1.3 | np |
| Disintegration | Report result | <1 min | 1′03″ |

np = not performed;
ND = not detectable

TABLE 42
Carglumic Acid 200 mg Tablets (60) packaged
in HDPE Bottle, stored at 5° C.

| Parameter | Specification | 0 M | 1 M |
|---|---|---|---|
| Hardness | Report result | np | np |
| Assay | 90.0% to 105.0% of label claim | 99.9 | 98.1 |
| Chromatographic | Imp. 6 RRT 1.20: ≤0.1% | ND | ND |
| Purity | Imp. 1 RRT 1.93: ≤0.1% | 0.03 | 0.03 |
| | Imp. 2 RRT 2.21: ≤0.1% | 0.01 | 0.01 |
| | Imp. 5a RRT 2.34: ≤0.1% | 0.01 | 0.01 |
| | Imp. 5b RRT 2.77: ≤0.1% | ND | ND |
| | Max, single unk. | 0.02 | 0.02 |
| | Impurity: ≤0.1% | | |
| | Total impurities: Report result | 0.1 | 0.1 |
| Dissolution | ≥85% in 15 min | 96 | np |
| | | (95-98) | |
| Loss on drying | Report result | 1.3 | np |
| Disintegration | Report result | <1 min | <1 min |

np = not performed;
ND = not detectable

TABLE 43
Carglumic Acid 200 mg Tablets (60) packaged
in HDPE Bottle, stored at 25° C./60% RH

| Parameter | Specification | 0 | 1 | 3 |
|---|---|---|---|---|
| Hardness | Report result | np | np | np |
| Assay | 90.0% to 105.0% of label | 99.9 | 98.3 | 100.2 |
| | claim | | | |
| Chroma- | Imp. 6 RRT 1.20: ≤0.1% | ND | ND | ND |
| tographic | Imp. 1 RRT 1.93: ≤0.1% | 0.03 | 0.04 | 0.02 |
| Purity | Imp. 2 RRT 2.21: ≤0.1% | 0.01 | 0.01 | 0.01 |
| | Imp. 5a RRT 2.34: ≤0.1% | 0.01 | 0.01 | 0.00 |
| | Imp. 5b RRT 2.77: ≤0.1% | ND | ND | ND |
| | Max, single unk. | 0.02 | 0.02 | ND |
| | Impurity: ≤0.1% | | | |
| | Total impurities: Report | 0.1 | 0.1 | 0.03 |
| | result | | | |
| Dissolution | ≥85% in 15 min | 96 | np | 99 |
| | | (95-98) | | (97-100) |
| Loss on drying | Report result | 1.3 | np | 0.7 |
| Disintegration | Report result | <1 min | 1′02″ | 1′09″ |

np = not performed;
ND = not detectable

TABLE 44
Carglumic Acid 200 mg Tablets (60) packaged
in HDPE Bottle, stored at 40° C./75% RH

| Parameter | Specification | 0 | 1 | 3 |
|---|---|---|---|---|
| Hardness | Report result | np | np | np |
| Assay | 90.0% to 105.0% of label | 99.9 | 97.9 | 100.2 |
| | claim | | | |
| Chroma- | Imp. 6 RRT 1.20: ≤0.1% | ND | ND | ND |
| tographic | Imp. 1 RRT 1.93: ≤0.1% | 0.03 | 0.07 | 0.31 |
| Purity | Imp. 2 RRT 2.21: ≤0.1% | 0.01 | 0.01 | 0.03 |
| | Imp. 5a RRT 2.34: ≤0.1% | 0.01 | 0.01 | 0.01 |
| | Imp. 5b RRT 2.77: ≤0.1% | ND | ND | ND |
| | Max, single unk. | 0.02 | 0.02 | 0.1 |
| | Impurity: ≤0.1% | | | |
| | Total impurities: Report | 0.1 | 0.1 | 0.6 |
| | result | | | |
| Dissolution | ≥85% in 15 min | 96 | np | 99 |
| | | (95-98) | | (99-100) |
| Loss on drying | Report result | 1.3 | np | np |
| Disintegration | Report result | <1 min | 2′50″ | 5′34″ |

np = not performed;
ND = not detectable

TABLE 45
Carglumic Acid 200 mg Tablets (60) packaged
in HDPE Bottle, stored at 30° C./65% RH

| Parameter | Specification | 0 M | 3 M |
|---|---|---|---|
| Hardness | Report result | np | np |
| Assay | 90.0% to 105.0% of label claim | 99.9 | 98.5 |
| Chromatographic | Imp. 6 RRT 1.20: ≤0.1% | ND | ND |
| Purity | Imp. 1 RRT 1.93: ≤0.1% | 0.03 | 0.03 |
| | Imp. 2 RRT 2.21: ≤0.1% | 0.01 | 0.00 |
| | Imp. 5a RRT 2.34: ≤0.1% | 0.01 | ND |
| | Imp. 5b RRT 2.77: ≤0.1% | ND | ND |
| | Max, single unk. | 0.02 | ND |
| | Impurity: ≤0.1% | | |
| | Total impurities: Report result | 0.1 | 0.04 |
| Dissolution | ≥85% in 15 min | 96 | 101 |
| | | (95-98) | (100-102) |
| Loss on drying | Report result | 1.3 | 0.8 |
| Disintegration | Report result | <1 min | 1′12″ |

np = not performed;
ND = not detectable

Further, longer stability tests were performed in HDPE bottles filled with 60 200 mg tablets as shown in Tables 46-49. These stability tests demonstrated a suitable behavior of the selected formulation at 200 mg strength when stored into a HDPE bottle filled with 60 tablets. Storage conditions up to 30° C./65% RH, based on the data available to-date, seem to be fully in compliance with the required specifications while, harsher conditions show values out of the specification starting from the 3rd month of stability.

TABLE 46
Carglumic Acid 200 mg Tablets (60) packaged in HDPE Bottle, stored at 5° C.

| Test | Shelf life specification | T 0 | 3 M | 6 M |
|---|---|---|---|---|
| Uniformity of | According to Ph. Eur 2.9.40 | L₁ < 15 | — | — |
| dosage units | | | | |
| Dissolution | Q ≥ 80% in 15 min | 98 | 99.4 | 99.2 |
| | | (97-101) | (96-102) | (98.2-101) |
| Assay | 90.0% to 105.0% of label claim | 99.8 | 98.6 | 99.6 |
| | Imp. 6 RRT 1.20: ≤0.1% | ND | ND | ND |
| | Imp. 1 RRT 1.93: ≤0.1% | ≤0.1 | 0.06 | 0.03 |
| | | (0.03) | | |
| | Imp. 2 RRT 2.21: ≤0.1% | ≤0.1 | 0.01 | 0.01 |
| | | (0.01) | | |
| | Imp. 5a RRT 2.34: ≤0.1% | ≤0.1 | 0.01 | 0.01 |
| | | (0.01) | | |
| | Imp. 5b RRT 2.77: ≤0.1% | ≤0.1 | ND | ND |
| | | (0.01) | | |
| | Max, single unk. Impurity: ≤0.1% | ≤0.1 | ND | ND |
| | | (0.02) | | |
| | Total impurities: ≤1.0% | ≤1.0 | 0.07 | 0.05 |
| | | (0.08) | | |
| Loss on | ≤5.0% | 1.1 | 1.2 | 1.5 |
| drying | | | | |
| Disintegration | NMT 3 min (15-25° C.) | 1′06″ | 1′28″ | 1′19″ |

ND = not detectable;
NMT = no more than

TABLE 47
Carglumic Acid 200 mg Tablets (60) packaged in HDPE Bottle, stored at 25° C./60% RH

| | Shelf life | | | | | | |
|---|---|---|---|---|---|---|---|
| Test | specification | 0 | 1 M | 3 M | 6 M | 9 M | 12 M |
| Uniformity of | According to Ph. Eur | L₁ < 15 | — | — | — | — | — |
| dosage units | 2.9.40 | | | | | | |
| Dissolution | Q ≥ 80% in 15 min | 98 | NP | 101 | 100 | 99 | Planned |
| | | (97-101) | | (99-104) | (99-101) | (98-100) | |
| Assay | 90.0% to 105.0% of | 99.8 | 99.4 | 95.7 | 99.8 | 99.7 | 99.9 |
| | label claim | | | | | | |
| | Imp. 6 RRT 1.20: ≤0.1% | ND | ND | ND | ND | ND | ND |
| | Imp. 1 RRT 1.93: ≤0.1% | ≤0.1 | 0.04 | 0.06 | 0.03 | 0.05 | 0.05 |
| | | (0.03) | | | | | |
| | Imp. 2 RRT 2.21: ≤0.1% | ≤0.1 | 0.01 | 0.01 | 0.01 | 0.01 | 0.01 |
| | | (0.01) | | | | | |
| | Imp. 5a RRT 2.34: ≤0.1% | ≤0.1 | 0.01 | ND | 0.02 | ND | 0.01 |
| | | (0.01) | | | | | |
| | Imp. 5b RRT 2.77: ≤0.1% | ≤0.1 | 0.01 | ND | ND | ND | ND |
| | | (0.01) | | | | | |
| | Max, single unk. | ≤0.1 | 0.02 | 0.02 | ND | ND | 0.06 |
| | Impurity: ≤0.1% | (0.02) | | | | | |
| | Total impurities: ≤1.0% | ≤1.0 | 0.11 | 0.11 | 0.05 | 0.06 | 0.16 |
| | | (0.08) | | | | | |
| Loss on drying | ≤5.0% | 1.1 | NP | 1.2 | 1.3 | 0.8 | 1.14 |
| Disintegration | NMT 3 min (15-25° C.) | 1′06″ | NP | 1′28″ | 1′30″ | 1′38″ | 1′15″ |

NP = not performed;
ND = not detectable;
NMT = no more than

TABLE 48
Carglumic Acid 200 mg Tablets (60) packaged in HDPE Bottle, stored at 30° C./65% RH

| | Shelf life | | | | | |
|---|---|---|---|---|---|---|
| Test | specification | 0 | 3 M | 6 M | 9 M | 12 M |
| Uniformity of | According to Ph. Eur | L₁ < 15 | — | — | — | — |
| dosage units | 2.9.40 | | | | | |
| Dissolution | Q ≥ 80% in 15 min | 98 | 99 | 98 | 97 | Planned |
| | | (97-101) | (98-100) | (97-100) | (88-101) | |
| Assay | 90.0% to 105.0% of | 99.8 | 95.9 | 98.4 | 100.1 | 100.0 |
| | label claim | | | | | |
| | Imp. 6 RRT 1.20: ≤0.1% | ND | ND | ND | ND | ND |
| | Imp. 1 RRT 1.93: ≤0.1% | ≤0.1 | 0.06 | 0.03 | 0.08 | 0.08 |
| | | (0.03) | | | | |
| | Imp. 2 RRT 2.21: ≤0.1% | ≤0.1 | 0.01 | 0.01 | 0.02 | 0.01 |
| | | (0.01) | | | | |
| | Imp. 5a RRT 2.34: ≤0.1% | ≤0.1 | 0.01 | 0.01 | ND | 0.01 |
| | | (0.01) | | | | |
| | Imp. 5b RRT 2.77: ≤0.1% | ≤0.1 | ND | ND | ND | ND |
| | | (0.01) | | | | |
| | Max, single unk. | ≤0.1 | 0.04 | ≤0.1 | ≤0.1 | 0.09 |
| | Impurity: ≤0.1% | (0.02) | | | | |
| | Total impurities: ≤1.0% | ≤1.0 | 0.12 | 0.05 | 0.3 | 0.3 |
| | | (0.08) | | | | |
| Loss on drying | ≤5.0% | 1.1 | 1.4 | 1.1 | 0.9 | 1.0 |
| Disintegration | NMT 3 min (15-25° C.) | 1′06″ | 1′43″ | 2′15″ | 2′44″ | |

ND = not detectable;
NMT = no more than

TABLE 49
Carglumic Acid 200 mg Tablets (60) packaged
in HDPE Bottle, stored at 40° C./75% RH

| Test | Shelf life specification | 0 | 1 | 3 | 6 |
|---|---|---|---|---|---|
| Uniformity of | According to Ph. Eur | L₁ < 15 | — | | |
| dosage units | 2.9.40 | | | | |
| Dissolution | Q ≥ 80% in 15 min | 98 | NP | 98 | 93 |
| | | (97-101) | | (96-99) | (90-96) |
| Assay | 90.0% to 105.0% of | 99.8 | 96.5 | 97.3 | 97.1 |
| | label claim | | | | |
| | Imp. 6 RRT 1.20: ≤0.1% | ND | 0.01 | 0.01 | ND |
| | Imp. 1 RRT 1.93: ≤0.1% | ≤0.1 | 0.15 | 0.21 | 0.75 |
| | | (0.03) | | | |
| | Imp. 2 RRT 2.21: ≤0.1% | ≤0.1 | 0.02 | 0.02 | 0.06 |
| | | (0.01) | | | |
| | Imp. 5a RRT 2.34: ≤0.1% | ≤0.1 | 0.01 | 0.02 | 0.02 |
| | | (0.01) | | | |
| | Imp. 5b RRT 2.77: ≤0.1% | ≤0.1 | 0.01 | 0.00 | 0.02 |
| | | (0.01) | | | |
| | Max, single unk. | ≤0.1 | 0.06 | 0.06 | 0.06 |
| | Impurity: ≤0.1% | (0.02) | | | |
| | Total impurities: ≤1.0% | ≤1.0 | 0.33 | 0.44 | 0.84 |
| | | (0.08) | | | |
| Loss on | ≤5.0% | 1.1 | NP | 1.5 | 1.6 |
| drying | | | | | |
| Disintegration | NMT 3 min (15-25° C.) | 1′06″ | NP | 5′41″ | 7′45″ |

NP = not performed;
ND = not detectable;
NMT = no more than

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A pharmaceutical formulation comprising:
(a) carglumic acid or a pharmaceutically acceptable salt thereof;
(b) tromethamine; and
(c) one or more pharmaceutically acceptable excipients.

2. The pharmaceutical formulation of claim 1, wherein carglumic acid or a pharmaceutically acceptable salt thereof and tromethamine are at a molar ratio of is from about 1:1 to about 1.5:1.

3. The pharmaceutical formulation of claim 2, wherein carglumic acid or a pharmaceutically acceptable salt thereof and tromethamine are at a molar ratio from about 1:1 to about 1.2:1.

4. The pharmaceutical formulation of claim 1, wherein carglumic acid or a pharmaceutically acceptable salt thereof and tromethamine are at a molar ratio of about 1.16:1.

5. The pharmaceutical formulation of claim 1, wherein the one or more pharmaceutically acceptable excipients is a filler.

6. The pharmaceutical formulation of claim 5, wherein the filler is selected from sugars, polyalcohols, amino acids, polymers, polysaccharides, inorganic salts, silica, or combinations thereof.

7. The pharmaceutical formulation of claim 5, wherein the filler is selected from glucose, mannose, maltose, sucrose, lactose, sorbitol, mannitol, maltitol, xylitol, glycine, polyvinylpyrrolidone (Crospovidone), Poly(l-vinylpyrrolidone-co-vinyl acetate) (Copovidone), dextran, sodium phosphate, potassium phosphate, sodium chloride, silicon dioxide, or combinations thereof.

8. The pharmaceutical formulation of claim 5, wherein the filler is sorbitol, mannitol, maltitol, crospovidone, copovidone, silicon dioxide, or combinations thereof.

9. The pharmaceutical formulation of claim 5, wherein the filler is Pharmaburst®.

10. The pharmaceutical formulation of claim 1, wherein the one or more pharmaceutically acceptable excipients is an effervescent agent.

11. The pharmaceutical formulation of claim 10, wherein the effervescent agent is selected from alkali metal bicarbonate, an alkaline earth metal bicarbonate, an alkali metal carbonate, an organic carbonate, or combinations thereof.

12. The pharmaceutical formulation of claim 10, wherein the effervescent agent is selected from ammonium bicarbonate, calcium bicarbonate, lithium bicarbonate, magnesium bicarbonate, potassium bicarbonate, sodium bicarbonate, arginine carbonate, ammonium carbonate, calcium carbonate, lysine carbonate, potassium magnesium carbonate, sodium carbonate, sodium glycine carbonate, sodium sesquicarbonate, zinc carbonate, or combinations thereof.

13. The pharmaceutical formulation of claim 10, wherein the effervescent agent is sodium carbonate, sodium bicarbonate, or combinations thereof.

14. The pharmaceutical formulation of claim 10, wherein the effervescent agent is sodium bicarbonate.

15. The pharmaceutical formulation of claim 1, wherein the one or more pharmaceutically acceptable excipients is a lubricant.

16. The pharmaceutical formulation of claim 15, wherein the lubricant is stearic acid, palmitic acid, calcium hydroxide, talc, corn starch, sodium stearyl fumarate, sodium stearate, magnesium stearate, zinc stearate, aluminum stearate, leucine, polyethylene glycol, glyceryl behenate, colloidal silicon dioxide, hydrogenated vegetable oil, mineral oil, or waxes.

17. The pharmaceutical formulation of claim 15, wherein the lubricant is magnesium stearate or sodium stearyl fumarate.

18. The pharmaceutical formulation of claim 15, wherein the lubricant is sodium stearyl fumarate.

19. The pharmaceutical formulation of claim 1, wherein the one or more pharmaceutically acceptable excipients is a sweetener.

20. The pharmaceutical formulation of claim 19, wherein the sweetener is selected from sugars or sugar alcohols.

21. The pharmaceutical formulation of claim 19, wherein the sweetener is selected from aspartame, ammonium glycyrrhizinate, sucralose, shaccarin sodium, sucrose, glucose, lactose, fructose, sorbitol, xylitol, or erythritol.

22. The pharmaceutical formulation of claim 19, wherein the sweetener is sucralose.

23. The pharmaceutical formulation of claim 1, wherein the one or more pharmaceutically acceptable excipients is a binder.

24. The pharmaceutical formulation of claim 23, wherein the binder is selected from celluloses, cellulose ethers, cellulose esters, tricalcium phosphate, povidone, copovidone, pregelatinized starch, dextrin, gelatin, maltodextrin, starch, zein, acacia, alginic acid, carbomers, cross-linked polyacrylates, polymethacrylates, sodium, guar gum, hydrogenated vegetable oil, magnesium aluminum silicate, or sodium alginate.

25. The pharmaceutical formulation of claim 23, wherein the binder is selected from methylcellulose, carboxymethylcellulose, hydroxypropyl cellulose, ethylcellulose, hydroxypropyl methylcellulose, or hydroxyethyl cellulose.

26. The pharmaceutical formulation of claim 23, wherein the binder is hydroxypropyl methylcellulose (HPMC).

27. The pharmaceutical formulation of claim 1, wherein the one or more pharmaceutically acceptable excipients is a wetting agent.

28. The pharmaceutical formulation of claim 27, wherein the wetting agent is selected from sucrose palmitate, polyethylene glycol-polypropylene glycol copolymer, metal alkyl sulfate, sodium lauryl sulfate, polyoxyethylene sorbitan fatty acid ester, polyoxyethylene alkyl ether, polyethylene glycol, polyoxyethylene castor oil derivatives, docusate sodium, quaternary ammonium amine compounds, sugar esters of fatty acids, polyethoxylated fatty acid esters, glycerides of fatty acids, and polyglycolized glycerides.

29. The pharmaceutical formulation of claim 27, wherein the wetting agent is polyethylene glycol or sucrose palmitate.

30. The pharmaceutical formulation of claim 27, wherein the wetting agent is sucrose palmitate.

31. The pharmaceutical formulation of claim 1, wherein the one or more pharmaceutically acceptable excipients is a glidant.

32. The pharmaceutical formulation of claim 31, wherein the glidant is selected from powdered cellulose, colloidal silicon dioxide, calcium silicate, magnesium trisilicate, talc, corn starch, or a combination thereof.

33. The pharmaceutical formulation of claim 31, wherein the glidant is colloidal silicon dioxide.

34. The pharmaceutical formulation of claim 1, wherein the formulation is in the form of a tablet.

35. The pharmaceutical formulation of claim 34, wherein the tablet has a disintegration time of less than 3 minutes.

36. The pharmaceutical formulation of claim 34, wherein the tablet has a disintegration time of less than or equal to 90 seconds.

37. The pharmaceutical formulation of claim 34, wherein the tablet has a dissolution profile characterized by at least 85% dissolution in pH 1.2 hydrochloric acid medium at 37° C.±0.5° C. in no more than 15 minutes as measured by high-performance liquid chromatography at 200 nm.

38. The pharmaceutical formulation of claim 34, wherein the tablet has a dissolution profile characterized by at least 85% dissolution in pH 4.5 sodium acetate buffer at 37° C.±0.5° C. in no more than 15 minutes as measured by high-performance liquid chromatography at 200 nm.

39. The pharmaceutical formulation of claim 34, wherein the tablet has a dissolution profile characterized by at least 85% dissolution in pH 6.8 potassium phosphate buffer at 37° C.±0.5° C. in no more than 15 minutes as measured by high-performance liquid chromatography at 200 nm.

40. The pharmaceutical formulation of claim 1, wherein the formulation comprises less than 0.5% of Impurity 1.

41. The pharmaceutical formulation of claim 1, wherein the formulation comprises less than 0.2% of Impurity 1.

42. The pharmaceutical formulation of claim 1, wherein the formulation comprises less than or equal to 0.10% of Impurity 1.

43. The pharmaceutical formulation of claim 1, wherein the formulation comprises less than or equal to 0.10% of Impurity 1 after the formulation is stored at 25° C. at 60% relative humidity (RH) for 3 months.

44. The pharmaceutical formulation of claim 1, wherein the formulation comprises less than or equal to 0.10% of Impurity 1 after the formulation is stored at 30° C. at 65% RH for 3 months.

45. A unit dose formulation comprising:
(a) about 100 mg to about 1000 mg of carglumic acid or a pharmaceutically acceptable salt thereof;
(b) tromethamine; and
(c) one or more pharmaceutically acceptable excipients.

46. The unit dose formulation of claim 45, comprising about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, or about 850 mg of carglumic acid or a pharmaceutically acceptable salt thereof.

47. The unit dose formulation of claim 45, comprising about 200 mg of carglumic acid or a pharmaceutically acceptable salt thereof.

48. The unit dose formulation of claim 47, wherein the unit dose comprises about 90 mg to about 150 mg of tromethamine.

49. The unit dose formulation of claim 47, wherein the unit dose comprises about 110 mg of tromethamine.

50. The unit dose formulation of claim 47, wherein the unit dose comprises about 100 mg to about 200 mg of Pharmaburst®.

51. The unit dose formulation of claim 47, wherein the unit dose comprises about 140 mg of Pharmaburst®.

52. The unit dose formulation of claim 45, comprising about 800 mg of carglumic acid or a pharmaceutically acceptable salt thereof.

53. The unit dose formulation of any one of claim 52, wherein the unit dose comprises about 400 mg to about 500 mg of tromethamine.

54. The unit dose formulation of any one of claim 52, wherein the unit dose comprises about 440 mg of tromethamine.

55. The unit dose formulation of claim 52, wherein the unit dose comprises about 500 mg to about 600 mg of Pharmaburst®.

56. The unit dose formulation of claim 52, wherein the unit dose comprises about 561 mg of Pharmaburst®.

57. A method of treating hyperammonaemia, comprising administering an effective amount of a pharmaceutical formulation of claim 1 to a patient in need thereof.

58. A method of treating hyperammonaemia, comprising administering an effective amount of a unit dose formulation of claim 45 to a patient in need thereof.
